(19) United States
(12) Patent Application Publication
(10) Pub. No.: US 2015/0164685 A1
(43) Pub. Date: Jun. 18, 2015

(54) PUPIL EXPANSION DEVICE

(71) Applicant: Suven Bhattacharjee, Kolkata (IN)

(72) Inventor: Suven Bhattacharjee, Kolkata (IN)

(21) Appl. No.: 14/628,636

(22) Filed: Feb. 23, 2015

Related U.S. Application Data
(63) Continuation-in-part of application No. 14/379,684.

(30) Foreign Application Priority Data
Feb. 27, 2013 (IN) 225/KOL/2013
Feb. 24, 2014 (IN) 228/KOL/2014
Aug. 13, 2014 (IN) 841/KOL/2014
Dec. 17, 2014 (IN) 1319/KOL/2014

(51) Int. Cl. A61F 9/007

(52) U.S. Cl. CPC A61F 9/007

(57) ABSTRACT

A device, providing enlargement and preventing collapse, of the pupil of the eye, during an ophthalmic surgical procedure. The device made of resiliently flexible material and configured as a ring, comprises plurality of notches at corners and flanges at sides. Notches engage the pupillary margin at different parts, pushing them apart, causing enlargement of the pupil. Consecutive flanges lie above and below the iris and bend the pupillary margin at the notches. The enlarged pupil allows a wide view of the structures deeper to the pupillary plane, previously obscured due to a small pupil. When the ring is manually expandable or self-expandable it is made of a combination of shape retaining and shape memory material that is selected from the group consisting of a self-enlarging material, a self-healing material, a self-reconfiguring programmable material, a biodegradable material, and/or drug-eluting material.

### CROSS REFERENCE TO RELATED APPLICATION

This application is a continuation-in-part of U.S. patent application Ser. No. 14/379,684, filed Aug. 19, 2014, which is a §371/U.S. National Phase of International Patent Application No. PCT/IN2013/000457, filed Jul. 23, 2013, and claims the benefit of (i) the aforementioned U.S. and international patent applications; (ii) Indian Provisional Patent Application No. 225/KOL/2013, filed Feb. 27, 2013, (iii) Indian Provisional Patent Application No. 228/KOL/2014, filed Feb. 24, 2014, (iv) Indian Provisional Patent Application No. 841/KOL/2014, filed Aug. 13, 2014, and (v) Indian Provisional Patent Application No. 1319/KOL/2014, filed Dec. 17, 2014. The disclosures of all of the foregoing patent applications are incorporated by reference herein in their entirety.

### BACKGROUND OF THE INVENTION

The present invention is in the field of ophthalmic surgery and relates to an improvement in the device for mechanical enlargement or dilation of the pupil of the eye during surgery.

During phacoemulsification surgery and vitreo-retinal surgery, when the pupil does not dilate with medicated drops, a device is required for mechanical enlargement of the pupil. Such a device has to maintain the pupil in the enlarged state and prevent it from collapsing for the entire duration of the surgery. Removal of the device returns the pupil to an unenlarged state to preserve its function and cosmesis. Eyes with non-dilating pupils are often also associated with floppiness of the iris, which poses additional difficulty during surgery.

In phacoemulsification surgery for cataract, a 1.6 to 2.8 mm incision in the side of the cornea is required to insert a phaco probe. Smaller incisions result in secure and astigmatically neutral wounds translating into better visual outcomes.

Vitreo-retinal surgery requires 0.6 mm or smaller incisions in the sclera to insert instruments into the eye. Since a corneal incision is not required, such an incision, only to insert a pupil-dilating device, should be as small as possible.

Current devices in use for pupillary dilation require a 2.2 to 2.5 mm incision for insertion into the eye. They snag the incision because of gaps or pockets at the corners, which have a biplanar structure with a top and bottom part. Such snagging makes removal of the device difficult and causes damage to the cornea. They require precise alignment to engage the pupillary margin into the small narrow wedge shaped gaps or pockets at the corners. This is particularly difficult because the surgeon has a top view and the device itself obscures view of the narrow gap, which is at the side of the device. The gaps or pockets have two structural planes having an upper and lower part making the corners thick and bulky. The gaps or pockets hold the pupillary margin and iris tissue passively and the pupillary margin can easily disengage during surgical manipulations.

Such continuous ring devices have been disclosed in U.S. Pat. No. 8,323,296, Dec. 4, 2012 by Malyugin, US Patent Application publication 2012/0269786, Nov. 15, 2012 by Dusek and US Patent Application publication 2013/0096386, Apr. 18, 2013 by Christensen & Colvard.

FIG. 1 shows an enlarged diagrammatic top perspective of the ring of the type disclosed in Malyugin U.S. Pat. No. 8,323,296, Dec. 4, 2012. The Malyugin ring 1 has a square configuration with four helical loops 2, 3, 4 and 5 formed by one complete turn of the strand and separated by sides 6, 7, 8 and 9. The two ends 10 and 11, of the ring have indented portions and are butt attached to each other by adhesive at the joint 12. Each loop has a wedge shaped gap 13 and 14, which faces the periphery of the ring, to receive and capture the iris tissue. The ring 1 maintains the pupil in an extended position while the central opening 15 provides a wide viewing area during surgery.

FIG. 2 shows an enlarged diagrammatic side view of ring of the type disclosed in Malyugin U.S. Pat. No. 8,323,296, showing iris tissue 16 within the wedge shaped gaps 13 and 14 of the loops. The side 9 connects the bottom part of gap 13, which is at one plane, to the top part of gap 14, which is at another plane.

FIG. 3 shows an enlarged diagrammatic top plan of the modified ring of the type disclosed in Dusek US Patent Publication 2012/0269786. The Dusek ring 17 has four sides 18, 19, 20 and 21. Side 20 has the end butt joint 22 where a drop of adhesive secures the ends 23 and 24. Side 19 is perpendicular to side 20. The sides are joined by a corner portion 25 which has three distinct bends, namely, a first obtuse bend 26 (essentially 135° inward and to the left as viewed in FIG. 3), second return bend 27 (essentially 180° inward and then down away from the viewer and then toward the right as viewed in FIG. 3), and a third obtuse bend 28 (essentially 135° up and to the left as viewed in FIG. 3). Corner portions 29, 30 and 31 are identical to corner portion 25.

FIG. 4 shows a perspective view of the ring of the type disclosed in Christensen & Colvard US Patent Publication 2013/0096386. In the described embodiment of this disclosure, ring 32 has a square formation with rounded corners 33, 34, 35 and 36. At each corner, there is a top plate 37, which forms generally one plane of the ring and there is a bottom plate 38, which forms generally a second plane of the ring. These planes are generally above and below the primary plane of the ring formed by connecting limbs 39, 40, 41 and 42. Together, the outer periphery of the top plate and the bottom plate at each corner form a lip feature, which is the entrance of the pockets 43, 44, 45, and 46 that contain a portion of the iris, which is supported in an open configuration.

Though discontinuous ring devices have been disclosed in U.S. Pat. No. 5,163,419, Nov. 17, 1992 by Goldman, U.S. Pat. No. 5,267,553, Dec. 7, 1993 by Graether, U.S. Pat. No. 6,620,098, Sep. 16, 2003 by Milverton and U.S. Pat. No. 6,648,819, Nov. 18, 2003 by Lee, these devices have not found acceptance because of larger incision size required, cumbersome manipulations involved and loose engagement.

A device, which can expand from a smaller contracted state, would be easier to place within the smaller non-dilated pupil. Such devices have been disclosed in U.S. Pat. Nos. 4,782,820, 5,299,564 & 5,441,045 and US patent application publication No. US 2013/0267988 A1. All these devices have an outward facing cup or groove which receives the pupil margin. This outward facing cup or groove has a ‘C’ shaped cross section aligned perpendicular to the general plane of the device. This results in a bulky device with a larger vertical profile. This is not desirable since space in the anterior chamber of the eye is limited. Moreover, these devices do not provide controlled engagement and enlargement of the pupil margin.

### OBJECTS OF THE INVENTION

A principal object of the present invention is to provide a device to enlarge the pupil of the eye, which requires a very small incision for insertion into the eye. Another object of the invention is to provide a device with a mechanism to engage the pupillary margin that will not snag the incision during insertion or removal. A further object of the invention is to provide a device, with an easier mechanism to engage the pupillary margin that does not require precise alignment of the pupillary margin into the narrow wedge shaped gaps or pockets at the sides of the device. A further object of the invention is to provide a device, with corners that can engage the pupillary margin but are slim and strictly in the same plane of the device. A further object of the invention is to provide a device, which not only enlarges the pupil, but also remains securely, yet reversibly fastened to iris tissue so that surgical manipulations do not lead to its disengagement. A further object of the invention is to provide a device that reduces floppiness of the Iris, which is often associated with non-dilating pupils.

Another object of the invention is to provide a device that requires a very small incision for insertion and removal. A further object of the invention is to provide a device that will not snag the incision during insertion or removal. A further object of the invention is to provide a device with corners that can engage the pupillary margin but are slim and generally in the same plane of the device.

Another principal object of the present invention is to provide a device that can engage the pupil margin in a small contracted state and then be expanded in a controlled manner to a larger expanded state enabling controlled expansion of the pupil. Another object of the invention is to provide a device that can be contracted to a smaller state to facilitate insertion of the device through a small incision. A further object of the invention is to provide a device that can be contracted to a smaller state for compact nesting within a delivery or extraction system. A further object of the invention is to provide a device that can be easily removed through a small incision. A further object of the invention is to provide a device that has a very thin profile.

Another principal object of the present invention is to provide a device that can engage the pupil margin in a small contracted state and then be expanded in a controlled manner to a larger expanded state enabling controlled expansion of the pupil. Another object of the invention is to provide a device that can be contracted to a smaller state to facilitate insertion of the device through a small incision. A further object of the invention is to provide a device that can be contracted to a smaller state for compact nesting within a delivery or extraction system. A further object of the invention is to provide a device that can be easily removed through a small incision. A further object of the invention is to provide a device that has a very thin profile.

### SUMMARY OF THE INVENTION

The present invention is a device to enlarge and prevent collapse, of the pupil of the eye, during surgery. The notches and flanges, on the same plane of a continuous or discontinuous ring, used to engage the pupillary margin, is the novelty of the present invention. The notches are at corners and alternate with side elements or flanges along the perimeter of the ring. The notches are open outwards with a blind receptacle inwards. Notches engage different parts of the pupillary margin and push them apart, resulting in sustained enlargement of the pupil. The flanges are formed from broad loops of the strand and are directed outwards.

The device is made of a strand of any resiliently flexible material. Thermally treated 5-0 nylon suture (0.10 to 0.12 mm) is such. Notches temporarily straighten as they pass through the incision allowing the device to be inserted through a very small incision. The device, having no gaps or pockets, being entirely disposed in a single plane, passes through the incision without snagging. The device bends the pupillary margin and iris at the notches and flanges, somewhat like a paper clip, creating a secure engagement. The iris being flexible, can tolerate such bending without any damage. The flanges lying in front of the iris reduce its floppiness by restricting the billowing effect.

Within the scope of the same inventive concept, variations in design are necessary, to allow the surgeon choices depending on the nature of the surgery, size of the eye, depth of the anterior chamber, associated co morbidity, size of incision, initial pupil size, desired pupil size etc. Variations in design are also necessary to suit different manufacturing capabilities.

The continuous form of the ring is in the form of a polygon with at least three sides and can be with or without a joint. The ends are joined by knotting or tying of the ends, or by chemical, thermal or ultrasonic bonding of the ends or by any other method. When made by molding, stamping or other methods there is no joint. The discontinuous form of the ring has at least three sides, two corners and four notches. This form requires a much smaller incision for insertion.

In one of the form of the invention, the ring has alternate side elements or flanges gently tilted backwards, enabling easy tucking of the flange under the iris.

Notches are formed by an inward loop of the strand at the corners or by an inward loop of the strand between two outward digit shaped protruded loops of the strand. In one form of the invention, the corners of the ring have two adjacent notches.

Positioning holes on the device help in manipulations of the device. When the device is made of expansible material, it enlarges to a larger size after insertion.

The ring device is configured to adapt one or more selected configurations from a folded, extended or deformed configuration, allowing insertion through small incisions.

The present invention further includes an expandable device to enlarge and prevent collapse of the pupil of the eye during surgery, wherein the novelty of the device is that it has a contracted state, multiple intermediate incrementally expanded states and a fully expanded state with pupil engaging parts aligned in the same general plane of the device resulting in a very thin profile. The device is made of a single strand or multiple pieces of single strand of resiliently flexible material. This strand may be solid or tubular. The device is a continuous ring formed either by interconnecting the two ends of a single strand or by interconnecting ends of the multiple pieces of single strand in series. The continuous ring forms a polygonal shape with at least three sides. At least one of the sides of the polygonal shape is expandable. Expansion of one or more connecting parts increases the circumference and the radial dimension of the device. Increase in radial dimension of the device causes the engaging parts to push the pupil margin radially away from the center.

The device has plurality of pupil margin engaging parts and supporting parts. The pupil margin engaging parts are at the corners of the polygonal shape and the supporting parts form the sides of the polygonal shape. The pupil margin engaging parts and the supporting parts are disposed in the same general plane of the device. The pupil margin engaging part is a notch, formed by a short acute inward bend towards the center of the ring, a return outward bend followed by a third acute bend of the strand. At least one supporting part has an interlocking slip joint and is expandable. In the form of the device made of a tubular strand, such a slip joint is a telescoping type. In the form of the device made of a solid strand the slip joint is a non-telescoping sliding joint type. In one form of the device, the ring is made of a single strand with first end and second end interconnected at a supporting part in a manner that they can slide in relation to each other to result in an expanded state of the ring. In another form of the device, the ring is made of multiple single strand units in series with interconnected ends at more than one supporting part that can slide in relation to each other to result in an expanded state of the ring. In this form of the device, all the interconnected units are disposed in the same general plane of the device. In this form of the device, the second end of one unit and the first end of the consecutive unit in the series are interconnected. In one preferred form of the device, the ring is made of four single strand units in series with interconnected ends and has a substantially square configuration.

In the form of the device made of single solid strand or multiple pieces of single solid strands in series, the strand has a circular cross section throughout except at the segment proximal to an end up to completion of the preceding notch. In this segment, immediately after the preceding notch the strand gradually flattens up to the end to form a thinner and broader leaf. In this segment, the cross section of the strand gradually changes from circular to elliptical to a narrow rectangle. Each leaf has a hole, gap or slit close to the end. At a sliding joint, the first end of a unit is threaded through the hole in the second end of the preceding unit and the second end of the preceding unit is threaded through the hole of the first end of the consecutive unit, resulting in an interlocking sliding joint. As the supporting part is expanded, the broader leaf may roll at the sides as it snugly fits in the hole of the other unit. Interconnecting the ends of consecutive units in such manner creates a continuous chain. Interconnecting the first end of the first unit and the second end of the last unit creates a continuous ring. A discontinuity created in the margin of the hole of either the first end of the first unit or the second end of the last unit allows this interconnection to be completed. After the continuous ring is formed, the discontinuity in the margin of the hole is closed with glue.

In the form of the device made of a tubular strand, the strand has a polygonal cross-section. The area of the polygonal cross section varies in different parts. A telescopic slip joint interconnects the ends at the expandable supporting part. The polygonal cross section restricts axial rotation of the strands at the joint. In one preferred form, the tubular strand has a square cross section. An inward flange or tapering of the outer tube and an outward flange or flaring of the inner tube limits the extent of expansion at the supporting part.

The present invention further includes an expandable device to enlarge the pupil of the eye during surgery. The novelty of the device is in the use of shape retaining pliant material, either wholly or partly in combination with resilient shape memory parts, to provide controlled expansion of the device from a deformed state to an undeformed state and vice versa. The shape memory resilient parts, when present, allow shape recovery and the shape retaining pliant parts allow controlled expansion of the device. The device comprises a strand formed as a ring at least partially enclosing a central space with plurality of pupil margin receiving parts connected by supporting parts shaped as flanges. The ring may be open or closed with at least three sides. When closed the ring forms a polygonal shape. The pupil margin engaging parts are at the corners and the supporting parts shaped as flanges are at the sides of the polygonal shape. In one embodiment of the invention, at least one of the pupil margin engaging parts at the corners is resiliently flexible and has shape memory and at least one of the supporting parts forming a flange, is pliant and shape retaining. Other embodiments have varying combinations of shape memory resiliently flexible pupil margin engaging parts at the corners and shape retaining supporting parts forming flanges. In another embodiment, the entire device is made of only shape retaining pliant material. Shape retaining and pliability properties of the pliant parts is such that manual instruments, automated instruments, other external or internal stimuli or forces can bend or straighten the pliant parts of the device inside or outside the eye but the constricting or dilating forces of the pupil of the eye cannot bend or straighten these parts. In other words, the shape retaining strength of the pliant parts is more than the constricting or dilating forces of the pupil margin. Each corner portion includes a blind end, a first limb connecting one flange to the blind end on one side, and a second limb connecting the next flange to the said blind end on an opposite side, to form a receiving means. In one embodiment, the blind end, the first limb and the second limb of a corner portion are all disposed in the same plane. In one embodiment, all corner portions are disposed in the same plane. In one embodiment, each flange is disposed entirely in a single plane. In one embodiment, all flanges are disposed in the same plane. In one embodiment, all corner portions and all flanges are disposed in the same plane. This embodiment has a very thin profile in the undeformed state. In different embodiments, in the contracted state, one or more pliant or resilient parts or both are bent or folded in a manner to reduce at least one radial dimension of the device. In some contracted states, the device remains in a thin single plane without any overlap of parts. In other contracted states, the parts of the device may overlap each other to reduce the radial dimensions of the device further. Such contracted states facilitate delivery of the device through a small incision or compact nesting of the device within a delivery system. The device in its expanded state is easily deformed to an elongated shape as it is removed from the eye by disengaging it from the pupil margin and pulling it out through a small incision. Alternatively, the device is removed through a small incision with or without an extraction device, in a contracted or expanded state.

In one embodiment of the invention, the resilient and pliant parts of the ring are continuous. In another embodiment of the invention, the resilient and pliant parts of the ring are seamlessly merged. In other embodiments, the ring may have one or more, fused, adhesive or any other joint between the resilient and pliant parts or within a resilient or pliant part.

The present invention further includes a self-expanding device to enlarge the pupil of the eye. The novelty of the device is in its slow self-expansion from a smaller size to a predetermined expanded size and shape. In its expanded state, the device comprises a ring with sides and corners, at least partially enclosing a central space and having notches and flanges. Corner portions are connectively disposed between the flanges and form receiving means for receiving a portion of the pupillary margin. The said corner portions extend into said central space. Each corner portion includes a blind end, a first limb connecting one flange to the blind end on one side, and a second limb connecting the next flange to the said blind end on an opposite side, to form a receiving means. The receiving means includes a notch sized and shaped to receive a portion of the pupillary margin such that the pupil margin and adjacent iris passes over one flange and first limb of the corner portion and under the second limb of the corner portion and the next flange. The device may have a closed or open ring configuration. The closed ring configuration has a polygonal shape. The device is either made of shape memory material, self-enlarging material or a combination of both. These self-expanding devices expand over a period of few seconds or minutes to reach their fully expanded state. The shape of the contracted smaller state of the device may or may not have resemblance to the fully expanded state of the device. The notches or flanges may not be discernible in the contracted smaller state of the device. The device when fully expanded has sufficient mechanical properties to keep the pupil expanded. The device may be made of solid or tubular filamentous material. The device may have one or more joints. The shape memory self-expanding material either spontaneously expands on being released into the eye or expands in response to a stimulus. Such stimulus could be thermal, hydrational, exposure to body fluids/physiological saline/balanced salt solution/viscoelastic material, ionic (change in pH), osmotic, chemical, electrical, magnetic, optical, electromagnetic or any other external or internal, contact or non-contact stimulus.

In one embodiment, the self-expanding device expands by moving from a first shape to a second shape, by shape memory effect. The first shape is small because of folding of one or more parts of the device. The second shape is larger than the first shape because of unfolding or straightening of part or whole of the device. The circumference of the ring of the second shape is same as that of the first shape. In this embodiment, the device is partly or completely made of one or more shape memory materials. These may be shape memory alloys, shape memory polymers, or any other material exhibiting shape memory effect or a combination of such materials. Such shape memory materials are also referred to as smart materials.

In another embodiment, the self-expanding device expands by moving from a first shape and size to a second shape and size, by shape memory effect as well as self-enlargement of part or whole of the device. The circumference of the ring of the second shape is larger than that of the first shape. Such a device is partly or wholly, made of shape memory hydrogels or other material capable of growth in size in response to stimuli or changes in environment. Such hydrogels are stimuli responsive, tough, low swelling and with mechanical properties which may be tuneable. These tough hydrogels may be slip-link network hydrogels, nanocomposite hydrogels, double network hydrogels, multifunctional crosslinked hydrogels, homogeneous hydrogels or hybrid ionic-covalent interpenetrating polymer network (IPN) hydrogels. These hydrogels may also be nanocomposite smart gels, DNA Hydrogels or Hybrid Hydrogels.

In another embodiment, the self-expanding device is made of a self-healing and/or self-reconfiguring programmable material with mechanical properties. The device is delivered into the eye in a solid, semisolid or liquid state. The device is delivered into the eye as a closed ring or an open ring. In one form of the device, the open ring forms a closed ring inside the eye, spontaneously or in response to external or internal stimuli or when the ends are approximated. In one form, the device is delivered as multiple pieces into the eye, and these pieces either spontaneously join or are joined together inside the eye to form an open or closed ring.

In one embodiment, the device is removed from the eye in its expanded size. In another embodiment, it is removed after contraction to a smaller size. In another embodiment, it is removed in smaller pieces. In another embodiment, it is removed after liquefaction or dissolving in a solvent.

In another embodiment, the self-expanding device is made of a biodegradable material, which is spontaneously degraded and removed from the eye after a defined period of minutes, hours, days or months. Such a device is useful when the pupil does not dilate with medications but frequent examination of the structures deeper to the pupillary plane is desired for diagnostic or therapeutic purposes. Such a device would be helpful in children with Retinopathy of Prematurity (ROP) or adults with Diabetic Retinopathy. Such a device eliminates the effort and complications associated with removal of the device.

In another embodiment, the self-expanding device is made of a biocompatible material designed as a long term prosthetic for prolonged expansion of the pupil over days, months, years or even permanently.

In another embodiment, the self-expanding biodegradable or non-biodegradable device, carries and delivers drugs into the eye (i.e., is formed from a drug-eluting material). The device may be loaded with one or more drugs at initial placement and/or be topped up with one or more drugs at intervals.

### BRIEF DESCRIPTION OF THE DRAWINGS

FIG. 1 is an enlarged diagrammatic top perspective of the ring of the type disclosed in Malyugin U.S. Pat. No. 8,323,296, Dec. 4, 2012.

FIG. 2 is an enlarged diagrammatic side view of ring of the type disclosed in Malyugin U.S. Pat. No. 8,323,296, showing iris tissue within the wedge shaped gaps of the loops.

FIG. 3 is an enlarged diagrammatic top plan of the modified ring of the type disclosed in Dusek US Patent Publication 2012/0269786.

FIG. 4 is a perspective view of the ring of the type disclosed in Christensen & Colvard US Patent Publication 2013/0096386.

FIG. 5 is an enlarged diagrammatic top view of one form of the device of the present invention, showing a square shaped continuous ring with a joint.

FIG. 6 is an illustration of the pupil maintained in an enlarged position by the device of FIG. 5.

FIG. 7 is an enlarged diagrammatic side view showing the relation of iris tissue to the notches.

FIG. 8 is an illustration of the insertion of the ring device into the eye and engagement of the pupillary margin with the first notch of the device of FIG. 5.

FIG. 9 is an illustration of the stretched pupillary margin after engagement by the second notch of the device of FIG. 5.

FIG. 10 is an illustration of the pupil fully enlarged after engagement by all four notches of the device of FIG. 5.

FIG. 11 is an enlarged diagrammatic top view of one form of the device of the present invention, showing a discontinuous ring with five sides, four corners and six notches.

FIG. 12 is an illustration of the pupil maintained in an enlarged position by the device of FIG. 11.

FIG. 13 is an enlarged diagrammatic top perspective of one form of the device of the present invention, showing alternate flanges of the device of FIG. 11, gently tilted backwards.

FIG. 14 is an enlarged diagrammatic top view of one form of the device of the present invention, showing plurality of positioning holes on a hexagon shaped continuous ring with no joint.

FIG. 15 is an enlarged diagrammatic top view of one form of the device of the present invention, showing engagement of the pupillary margin by notches formed by an inward loop of the strand between two outward digit shaped protruding loops of the strand of a square shaped continuous ring with no joint.

FIG. 16 is an enlarged diagrammatic top view of one form of the device of the present invention, showing engagement of the pupillary margin by two adjacent notches at the corners of a square shaped continuous ring with no joint.

FIG. 17 is an enlarged diagrammatic top view of another form of the device of the present invention made of four solid single strand units, showing a square shaped continuous ring in its contracted state with four pupil-engaging parts or notches and four expandable supporting parts with interlocking sliding joints.

FIG. 18 is an enlarged diagrammatic top view of the device of FIG. 17 showing the square shaped continuous ring in its expanded state.

FIG. 19 is a diagrammatic view showing a unit of the device of FIG. 17 and its cross sections at different parts.

FIG. 20 is a diagrammatic view of a step in forming the interlocking sliding joint of the device of FIG. 17 showing the first end of a unit being threaded through the hole of the second end of the preceding unit.

FIG. 21 is a diagrammatic view of another step in forming the interlocking sliding joint of the device of FIG. 17 showing first end of a unit being threaded through the hole of the second end of the preceding unit and first end of the preceding unit being threaded through the hole of the first end of the consecutive unit.

FIG. 22 is a diagrammatic view of the final step in forming the interlocking sliding joint of the device of FIG. 17 showing two consecutive units of the device of FIG. 17 interconnected at an interlocking sliding joint at the supporting part.

FIG. 23 is an enlarged diagrammatic top view of the telescopic sliding joint at the supporting part of one form of the device of the present invention made of a tubular strand with square cross section.

FIG. 24 is a diagrammatic longitudinal section through the telescopic interlocking sliding joint of the device of FIG. 23 showing an inward flange of the outer tube and an outward flange of the inner tube.

FIG. 25 is a top view of the device of FIG. 17, in use in a non-dilated pupil of the eye.

FIG. 26 is a top view of the device of FIG. 17, in an expanded state in an enlarged, or dilated, pupil.

FIG. 27 is an enlarged diagrammatic top perspective of a square closed ring of the present invention in its expanded undeformed state with shape memory resilient notches and shape retaining pliant flanges.

FIG. 28 is an enlarged diagrammatic top perspective of a hexagonal closed ring of the present invention in its expanded undeformed state with shape memory resilient notches and shape retaining pliant flanges.

FIG. 29 is an enlarged diagrammatic top view of a contracted state of the device of FIG. 27, with all four notches and all four flanges deformed.

FIG. 30 is an enlarged diagrammatic top perspective view of a contracted state of the device of FIG. 27, compactly nested in a delivery device, with all four notches and all four flanges deformed.

FIG. 31 is an enlarged diagrammatic top view of a contracted state of the device of FIG. 27, after expulsion from the delivery device of FIG. 30, with all four notches undeformed and all four flanges still deformed.

FIG. 32 is an enlarged diagrammatic top view of the device of FIG. 27, being enlarged manually from the contracted state of FIG. 31, with two Kuglen hooks.

FIG. 33 is an enlarged diagrammatic top view of a contracted state of the device of FIG. 27, with all four notches and only two flanges deformed.

FIG. 34 is an enlarged diagrammatic top view of a contracted state of the device of FIG. 27, after expulsion from a delivery device, after having been in a contracted state of FIG. 33, with all four notches undeformed and two flanges deformed.

FIG. 35 is an enlarged diagrammatic top view of a contracted state of the device of FIG. 27, with two Kuglen hooks, causing deformation of the two previously undeformed flanges of FIG. 34, to enable the notches, receive the non-dilating pupil margin.

FIG. 36 is an enlarged diagrammatic top view of a contracted state of the device of FIG. 27, with deformation of two flanges as in FIG. 35, and use of Kuglen hooks to straighten the loops of the other two flanges, to expand the device and cause expansion of the pupil.

FIG. 37 is an enlarged diagrammatic top perspective of one embodiment of the expanded state of a self-expanding square closed ring of the present invention made of shape memory material.

FIG. 38 is an enlarged diagrammatic top perspective of one embodiment of the expanded state of a self-expanding hexagonal closed ring of the present invention made of shape memory material.

FIG. 39 is an enlarged diagrammatic top view of the device of FIG. 37 showing the contracted state.

FIG. 40 is an enlarged diagrammatic top perspective view of the device of FIG. 39, compactly nested in a delivery device.

FIG. 41 is an enlarged diagrammatic top view of the device of FIG. 37, in a partially self-expanded state.

FIG. 42 is an enlarged diagrammatic top view of another embodiment of the contracted state of the device of FIG. 37.

FIG. 43 is an enlarged diagrammatic top view of the device of FIG. 42, compactly nested within a tubular sleeve.

FIG. 44 is an enlarged diagrammatic top view of the device of FIG. 42, in nearly fully self-expanded state.

FIG. 45 is an enlarged diagrammatic top view of a contracted state of a device of the present invention made of shape memory self-enlarging material.

FIG. 46 is an enlarged diagrammatic top view of the fully expanded state of the device of FIG. 45.

FIG. 47 is an enlarged diagrammatic top view of a contracted and open ring state of a device of the present invention made of self-enlarging, self-healing and self-reconfiguring programmable material.

FIG. 48 is an enlarged diagrammatic top view of a fully expanded and closed ring state of a device of the present invention formed by self-enlargement, self-healing and self-reconfiguration of the device of FIG. 47.

FIG. 49 is an enlarged perspective view of an alternative embodiment of the invention.

FIG. 50 is an enlarged perspective view of another alternative embodiment of the invention.

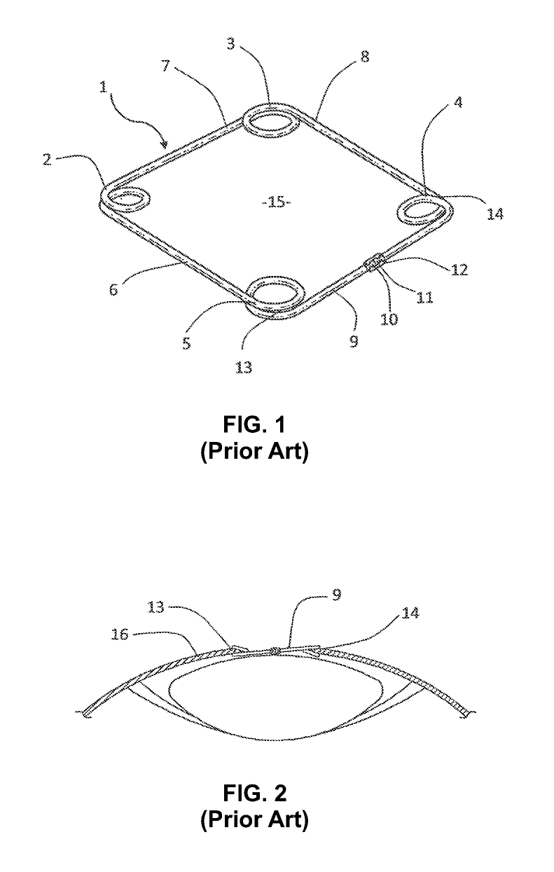

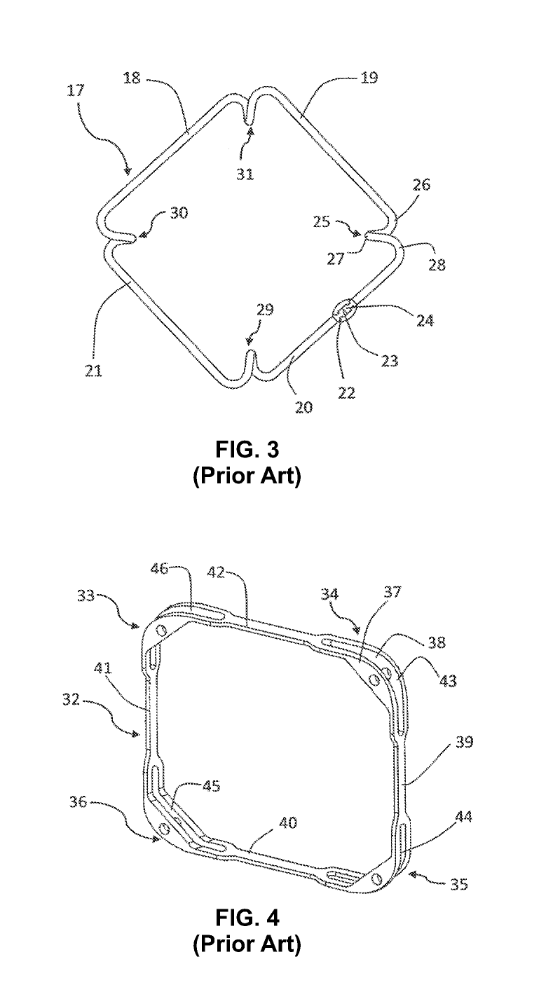

### DETAILED DESCRIPTION OF THE INVENTION

The relevant features of existing devices and the novelty of the present invention, is illustrated in the accompanying drawings, throughout which, like reference numerals indicate corresponding parts in the various figures.

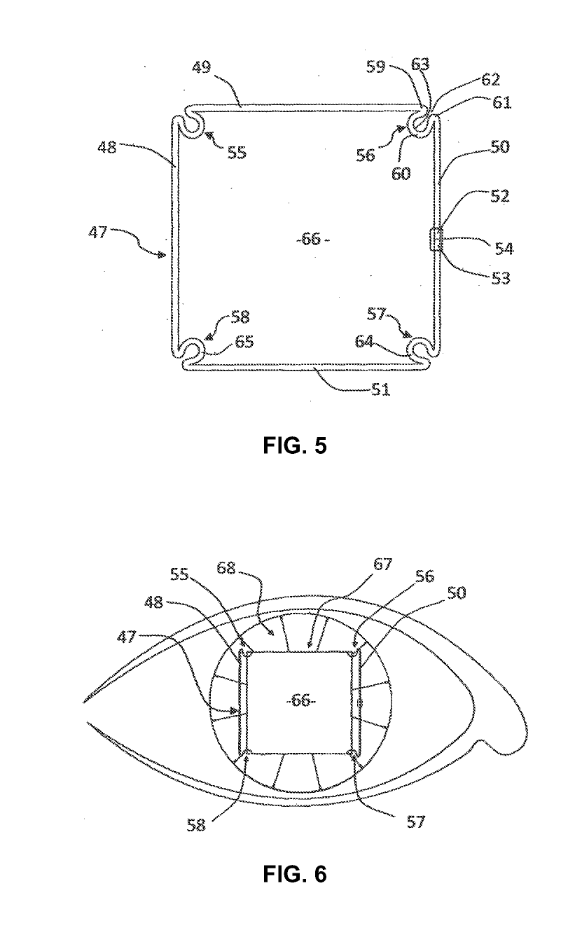

FIG. 5 shows an enlarged diagrammatic top view of the ring of the present invention used for enlargement and prevention of collapse of the pupil. The ring 47 is made of any resiliently flexible strand, for example, thermally treated 4-0 nylon suture. It is a continuous ring, has a square configuration and has four sides 48, 49, 50 and 51, joined by corner portions. Side 50 has ends 52 and 53 joined at the butt joint 54 with adhesive. Each one of the corner portions 55, 56, 57 and 58, forms a notch, same numerals representing the notches. Corner portion 56 joins side 49 to side 50, which are perpendicular to each other. At the corner portion 56, the strand loops inwards to form a notch. Three distinct bends of the strand in the same plane form the notch. A first acute bend 59, second rounded return bend 60, and a third acute bend 61. The notch has a narrow outward opening 62, which allows iris tissue to enter the notch. Inwards, the notch has a blind, bulbous receptacle 63, which engages the pupillary margin and iris tissue gently. Corner portions 55, 57 and 58 are identical to corner portion 56. Parts 64 and 65 of corner portions 57 and 58 respectively, along with side 51, form an outward flange. Sides 48, 49 and 50, form similar flanges. The ring comprises alternate notches and flanges, all being in the same plane and enclosing a space 66.

FIG. 6 shows an illustration of the pupil maintained in an enlarged position by the ring 47 of FIG. 5. The notches at corners 55, 56, 57 and 58, engage the pupillary margin 67 at different parts and push them apart, causing enlargement of the pupil. The flanges at sides 48 and 50 remain in front of the Iris 68. The flanges at sides 49 and 51 remain behind the Iris and are not visible. The alternate notches and flanges cause bending of the pupillary margin and iris tissue somewhat like a paper clip. This results in a secure yet reversible engagement. The central opening 66, allows wide view of the structures deeper to the pupillary plane.

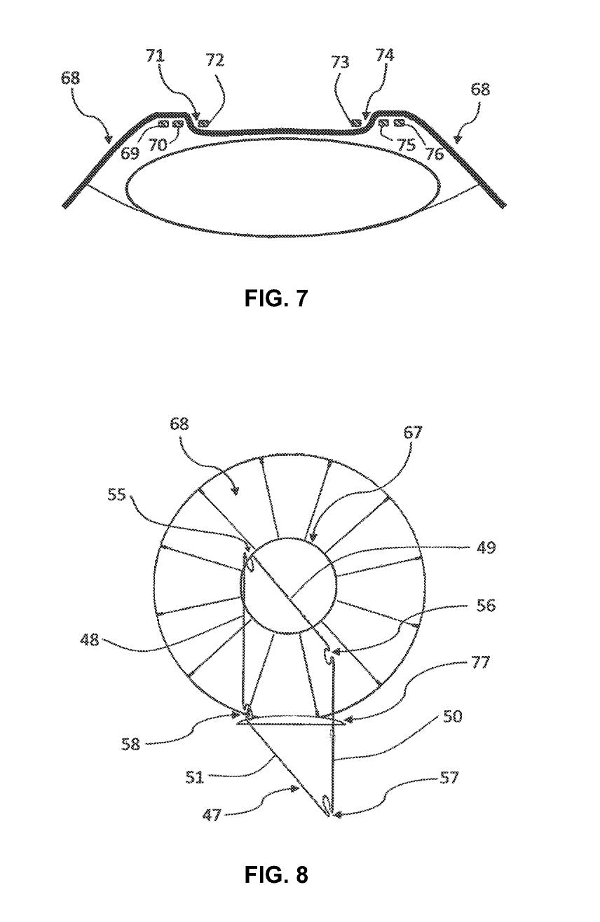

FIG. 7 shows an enlarged diagrammatic side view of the relation of iris tissue to the notches. This side view is at a vertical plane passing through the middle of any two adjacent notches of FIG. 6. The device distinctly bends the iris tissue 68, four times, as it passes through the two notches. From the left as viewed in FIG. 7, the iris 68 passes above the side element 69 and outer limb 70 of the notch 71. The first bend is at an obtuse angle as it passes downwards around the outer limb 70 of the notch 71 and through the notch. The second bend is at an obtuse angle to pass under the inner limb 72 of the notch 71. Iris 68 then makes a third bend at an obtuse angle as it passes upwards around the inner limb 73 of the notch 74 and through the notch. The final fourth bend is at an obtuse angle to pass above the outer limb 75 of the notch 74 and side element 76. As viewed in FIG. 7, side element 69, outer limb 70 of the notch 71, the notch 71, inner limb 72 of the notch 71, inner limb 73 of the notch 74, the notch 74, outer limb 75 of the notch 74 and side element 76, all lie in the same plane.

FIG. 8 refers to the usage of the invention, is an illustration of the insertion of the ring 47 of FIG. 5, into the eye and engagement of the pupillary margin 67 with the first notch 55 of the ring. A forceps (not shown here) carries the device or an injector (not shown here) delivers the device through the incision 77, into the anterior chamber of the eye. The flexible square ring 47 adopts a rhomboid configuration as it negotiates through a much smaller incision 77. The notches 56 and 58 open up and temporarily straighten out as the device passes through the incision. The leading first notch 55 hooks and engages the pupillary margin 67 pushing it in an outward direction. The pupillary margin 67 is lifted with a Hirschman hook (not shown here) to tuck the flange 49 under the pupillary margin 67 and iris 68.

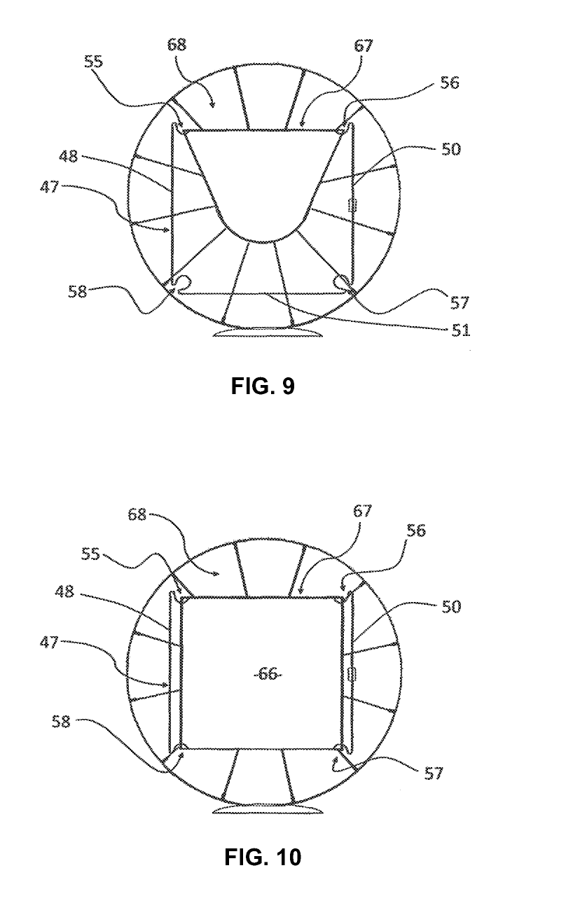

FIG. 9 refers to the usage of the invention in further detail, is an illustration of the stretched pupillary margin after engagement by the second notch 56 of the device 47 of FIG. 5. The resiliently flexible ring 47 has now returned to its square configuration. Notches 55 and 56 have engaged the pupillary margin 67 at two different points and pushed them apart. Flange 49 (not visible here), remains tucked under the pupillary margin 67, while flanges 48, 50 and 51, remain in front of the iris 68. As the pupillary margin is hooked again with an iris or hirschman hook, introduced through a side port (not shown here), a forceps introduced through another side port (not shown here), holds the flange 51 and tucks it under the pupillary margin 67 and iris 68.

FIG. 10 refers to the usage of the invention in further detail, is an illustration of the pupillary margin 67, fully enlarged and adopting a square configuration after engagement by all four notches 55, 56, 57 and 58 of the device 47 of FIG. 5. The flanges 48 and 50 remain in front of the Iris 68. The flanges at sides 49 and 51 remain behind the iris and are not visible. The central opening 66, allows wide view of the structures deeper to the pupillary plane. On completion of surgery, the device is easily disengaged from the pupillary margin and pulled out with a forceps. The device exits the eye without snagging the incision because notches are in the same plane as the flanges and are capable of straightening temporarily.

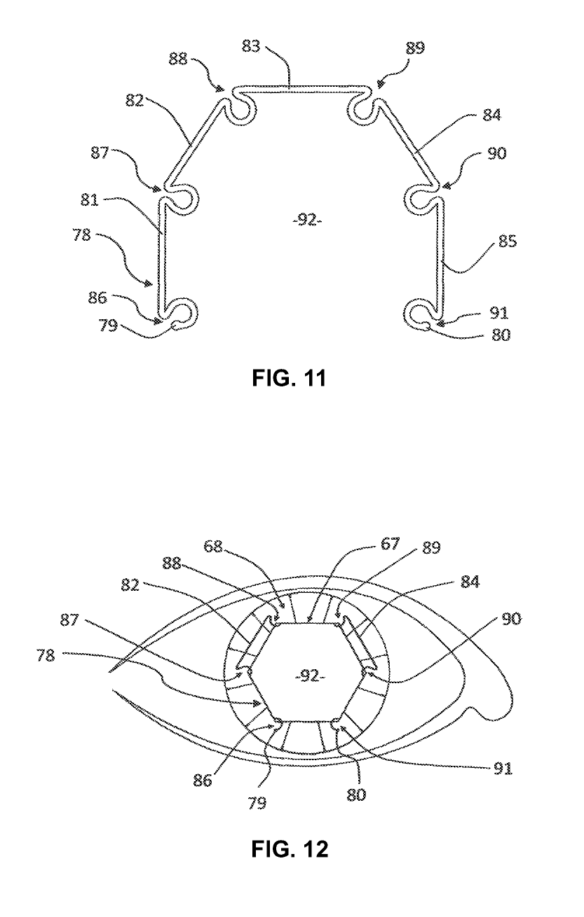

FIG. 11 is an enlarged diagrammatic top view of another form of the device of the present invention, showing a discontinuous ring 78 with ends 79 and 80, which are blunt or olive shaped to prevent damage to delicate structures of the eye. The discontinuous ring 78 has five sides 81, 82, 83, 84 and 85, which are shaped like flanges. Corner portions joining these sides are internally obtuse angled. The four corner portions 87, 88, 89 and 90, form four notches, same numerals representing the notches. Notch 86 is at the first end and notch 91 is at the second end of the ring. In the device of FIG. 11, the angles at corner 87 and 90 are equal to each other and the angles at corner 89 and 90 are equal to each other. The first side 81 and the fifth side 85 are parallel to each other, giving the device a flat top house shape. The central space 92, is closed on five sides by the device, and open on one side. In another form of the device of FIG. 11 (not shown here), the first side 81 and the fifth side 85, are unparallel, such that the distance between the ends 79 and 80, is more than that between the first corner 87 and fourth corner 90, giving the device the shape of a flat top tower.

FIG. 12 is an illustration of the pupil maintained in an enlarged position by the device of FIG. 11. The notches 86, 87, 88, 89, 90 and 91, engage the pupillary margin 67 at different parts and push them apart, causing enlargement of the pupil. Ends 79 and 80, and flanges 82 and 84 remain in front of the Iris 68. The flanges 81, 83 and 85 remain behind the Iris and are not visible. The constricting force of the pupil draws the notches at the ends 79 and 80 of the discontinuous ring closer and the resultant shape of the central space 92 of the device and that of the pupil is a hexagon. The central space 92, allows wide view of the structures deeper to the pupillary plane. While the device of FIG. 11 may be inserted into the eye, in the manner described above for the device of FIG. 5, the device may alternatively be inserted end first, into the eye, through a much smaller side port incision. The entire device is inserted into the anterior chamber and placed on the iris. The pupillary margin is hooked with an iris or hirschman hook, introduced through one side port incision, while a forceps introduced through another side port, holds flange 83 and tucks it under the pupillary margin 67. Similarly, flanges 81 and 85 are tucked under the pupillary margin.

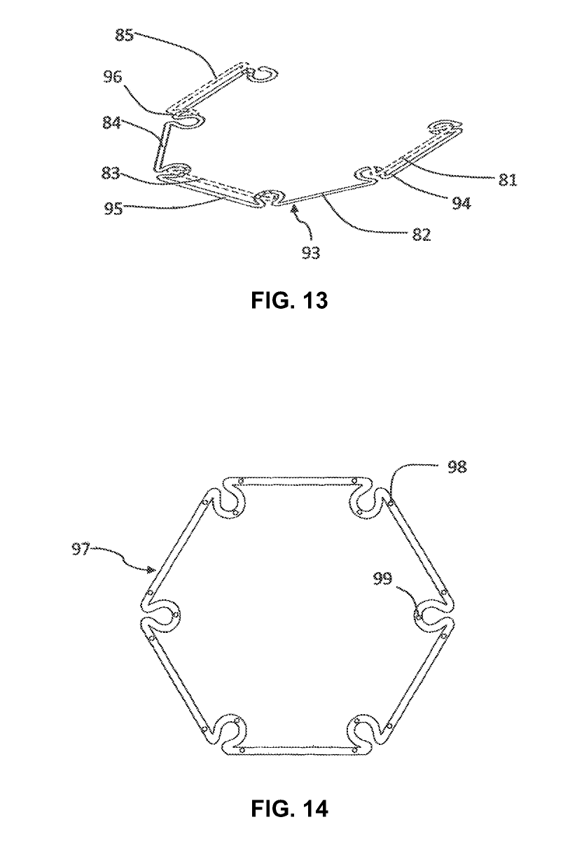

FIG. 13 is an enlarged diagrammatic top perspective of one form of the device of the present invention, showing alternate flanges of the device of FIG. 11, gently tilted backwards. Device 93 allows easier tucking of the flanges under the pupillary margin. Flanges 81, 83 and 85, shown in dotted lines, represent the previous straight position of flanges. Flanges 94, 95 and 96 represent the backward tilted flanges, respectively. The flanges are tilted all the way up to the centre of the notch or only at the peripheral edge. The position of flanges 82 and 84, which remain in front of the pupillary margin, is unaltered. Although tilted flanges are shown on the device of the present invention of FIG. 11, it is understood that such flanges may be present on all the forms of the device.

FIG. 14 is an enlarged diagrammatic top view of one form of the device of the present invention, showing plurality of positioning holes on a hexagon shaped continuous ring with no joint. Holes 98 and 99 are shown on a flange and notch respectively on the device 97. These holes are partial thickness or full thickness. These holes allow easy manipulation of the device inside the eye with the help of a pointed instrument called dialler. Although positioning holes or eyelets are shown on the device of the present invention of FIG. 14, it is understood that such positioning holes or eyelets may be present on all the forms of the device.

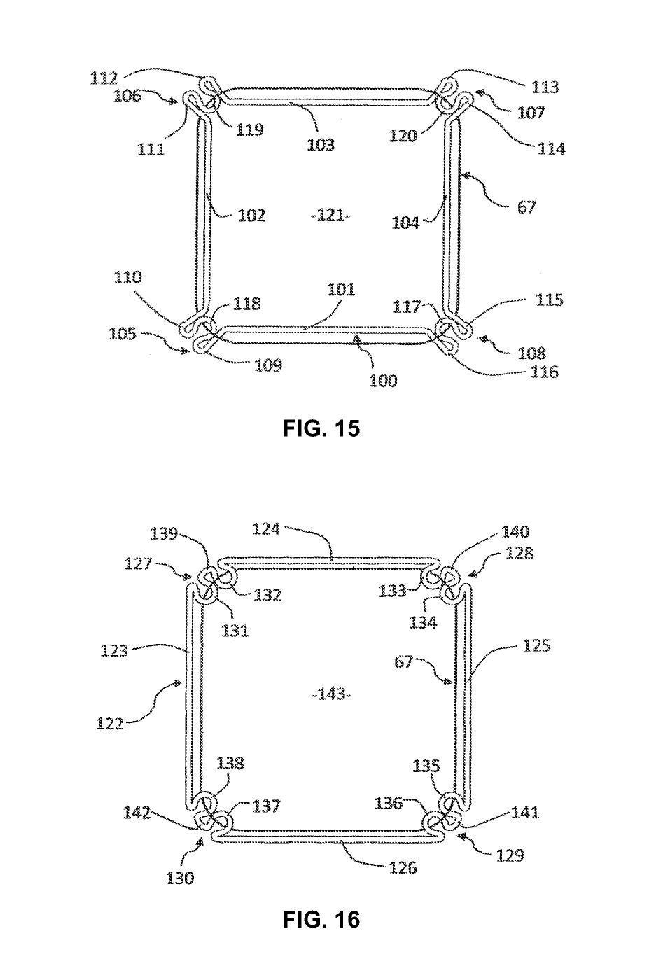

FIG. 15 is an enlarged diagrammatic top view of one form of the device of the present invention, showing engagement of the pupillary margin 67 by notches formed by an inward loop of the strand between two outward digit shaped protruding loops of the strand of a square shaped continuous ring with no joint. The ring 100 has four side elements 101, 102, 103 and 104. Corner portions 105, 106, 107 and 108, join the side elements. Corner portion 108 joins side 101 to side 104, which are perpendicular to each other. At the corner portion 108, the strand makes three distinct loops in the same plane to form a notch. Loops 115 and 116 are outward digit like protrusions. Notch 117, is formed by an inward loop between these two outward looped protrusions. Corner portions 105, 106 and 107 are identical to corner portion 108 and form notches 118, 119 and 120 respectively. The pupillary margin 67 has a square configuration as it is engaged by the notches 117, 118, 119 and 120, as it passes behind the elements 110, 111, 114 and 115, and in front of elements 112, 113, 116 and 109. The enclosed space 121, allows wide view of the structures deeper to the pupillary plane.

FIG. 16 is an enlarged diagrammatic top view of one form of the device of the present invention, showing engagement of the pupillary margin 67 by two adjacent notches at the corners of a square shaped continuous ring with no joint. The ring 122 has four side elements 123, 124, 125 and 126. Corner portions 127, 128, 129 and 130, join the side elements. Corner portion 127 joins side 123 to side 124, which are perpendicular to each other. At the corner portion 127, the strand makes three distinct loops in the same plane to form two adjacent paired notches or a double notch. Paired notches 131 and 132 are formed by two loops directed inwards. A digit like structure 139, is formed between these two notches by a loop directed outwards. Corner portions 128, 129 and 130, are identical to corner portion 127 and form paired notches 133, 134 and 135, 136 and 137, 138 respectively. The pupillary margin 67 has a square configuration, engaged by the notches 131, 132, 133, 134, 135, 136, 137 and 138. The pupillary margin 67 passes behind side elements 123, 124, 125 and 126, and in front of elements 139, 140, 141 and 142. The enclosed space 143, allows wide view of the structures deeper to the pupillary plane. The pupillary margin 67 could alternately pass (not shown here) in front of side elements 123, 124, 125 and 126, and behind elements 139, 140, 141 and 142.

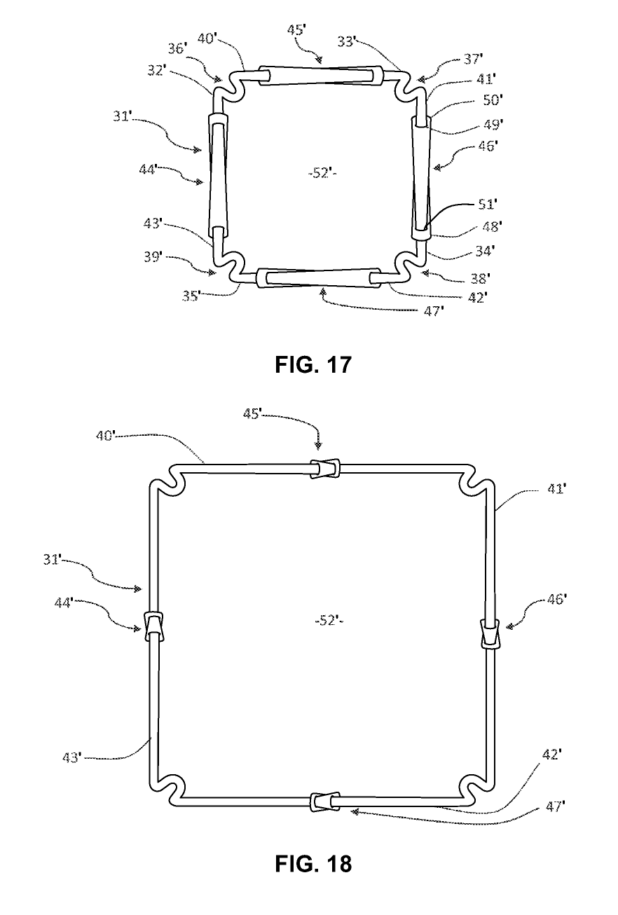

FIG. 17 shows an enlarged diagrammatic top view of one form of the device of the present invention in its contracted state. The device 31′ is a square shaped continuous ring entirely disposed in a single plane made of resiliently flexible material comprising four solid single strand units 32′, 33′, 34′ and 35′. The device has four pupil-engaging notches (loops) 36′, 37′, 38′ and 39′ and four expandable supporting parts 40′, 41′, 42′ and 43′ with interlocking sliding joints 44′, 45′, 46′ and 47′. At the interlocking sliding joint 46′ the second end 48′ of unit 33′ passes through the hole 49′ in the first end 50′ of unit 34′ and the first end 50′ of unit 34′ passes through the hole 51′ in the second end 48′ of unit 33′. The device 31′ encloses a space 52′.

FIG. 18 is an enlarged diagrammatic top view of the device 31′ of FIG. 17 showing the square shaped continuous ring in its expanded state. Supporting parts 40′, 41′, 42′ and 43′ have expanded because of sliding between the second end of the preceding unit and the first end of the consecutive unit at the interlocking joints 44′, 45′, 46′ and 47′. Because of expansion of the supporting parts, there is an increase in the circumference of the device, increase in the radial dimension of the device and increase in the size of the enclosed space 52′. The interlocking arrangement at the joints limits the expansion of the supporting parts and hence limits the size of the expanded state of the device.

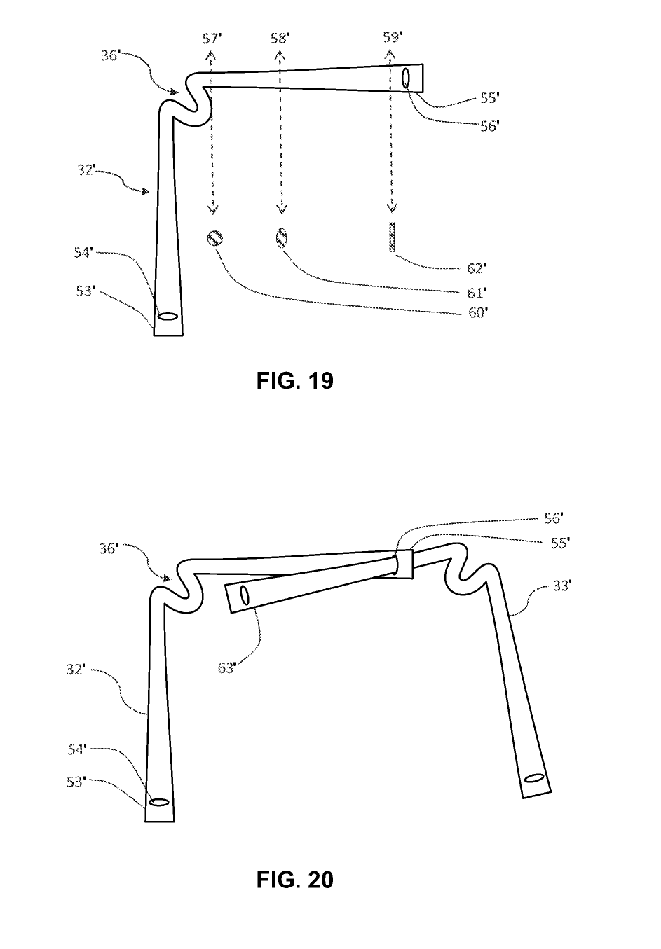

FIG. 19 is a diagrammatic view of unit 32′ of the device 31′ of FIG. 17. The unit has a first end 53′ with a hole 54′, the pupil engaging notch 36′ and the second end 55′ with a hole 56′. After completion of the notch 36′, the strand gradually flattens into a broader and thinner leaf. The cross sections at planes 57′, 58′ and 59′ are round 60′, ellipse 61′ and narrow rectangle 62′ respectively.

FIG. 20 is a diagrammatic view of the first step in forming the interlocking sliding joint of the device of FIG. 17, showing two consecutive units 32′ and 33′ of the device 31′. The first end 63′ of unit 33′ is threaded through the hole 56′ of the second end 55′ of the preceding unit 32′.

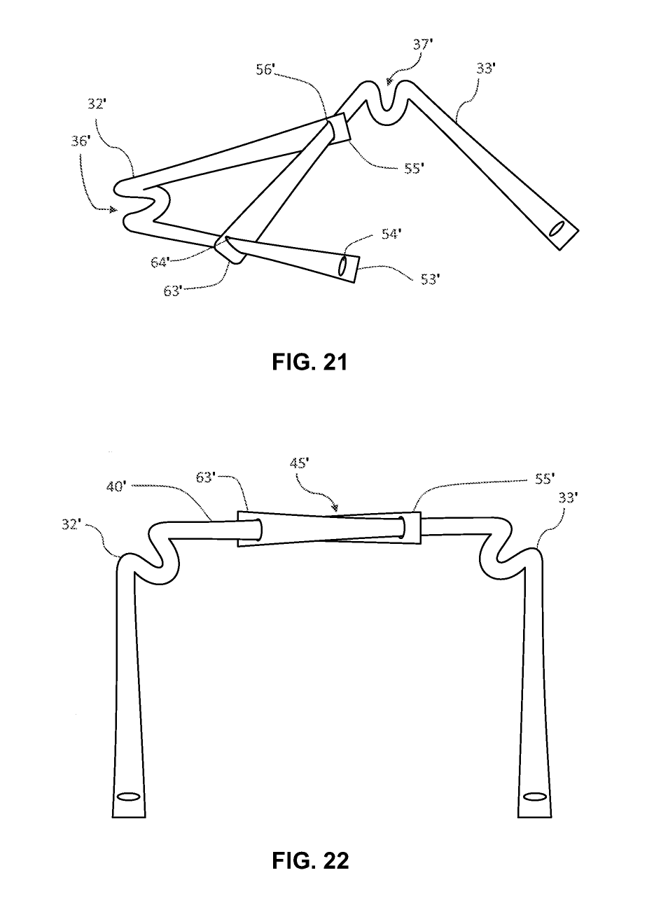

FIG. 21 is a diagrammatic view of the next step in forming the interlocking sliding joint of the device of FIG. 4 showing two consecutive units 32′ and 33′ of the device 31′. As shown in FIG. 20 the first end 63′ of unit 33′ is threaded through the hole 56′ of the second end 55′ of the preceding unit 32′. Then the first end 53′ of unit 32′ is bent towards its second end 55′ and threaded through the hole 64′ of the first end 63′ of the consecutive unit 33′.

FIG. 22 is a diagrammatic view of the final step in forming the interlocking sliding joint of the device of FIG. 17 showing consecutive units 32′ and 33′ of the device 31′. The interlocking sliding joint 45′ at the supporting part 40′ connects the second end 55′ of unit 32′ to the first end 63′ of unit 32′. The supporting part 40′ expands as units 32′ and 33′ are drawn apart bringing the ends 55′ and 63′ close together.

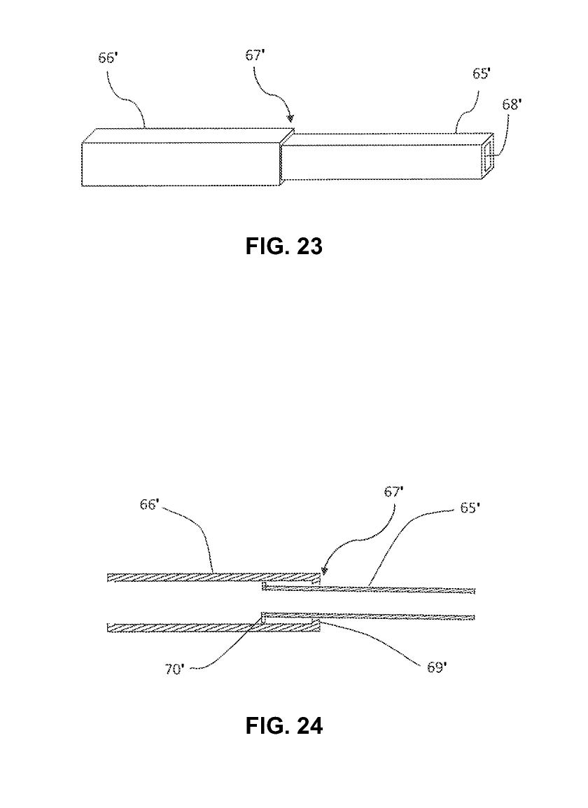

FIG. 23 is an enlarged diagrammatic view of a telescopic slip joint of one form of the device of the present invention. The inner tube 65′ telescopes into the outer tube 66′ at joint 67′. The inner tube 65′ has a square cross section 68′ with area less than that of the outer tube 66′, which also has a square cross section. The square cross section prevents axial rotation of the tubes.

FIG. 24 is a diagrammatic longitudinal section through the telescopic interlocking slip joint of the device of FIG. 23. The outer tube 66′ has an inward flange 69′ and the inner tube 65′ has an outward flange 70′, which restricts the limit of expansion of the supporting part.

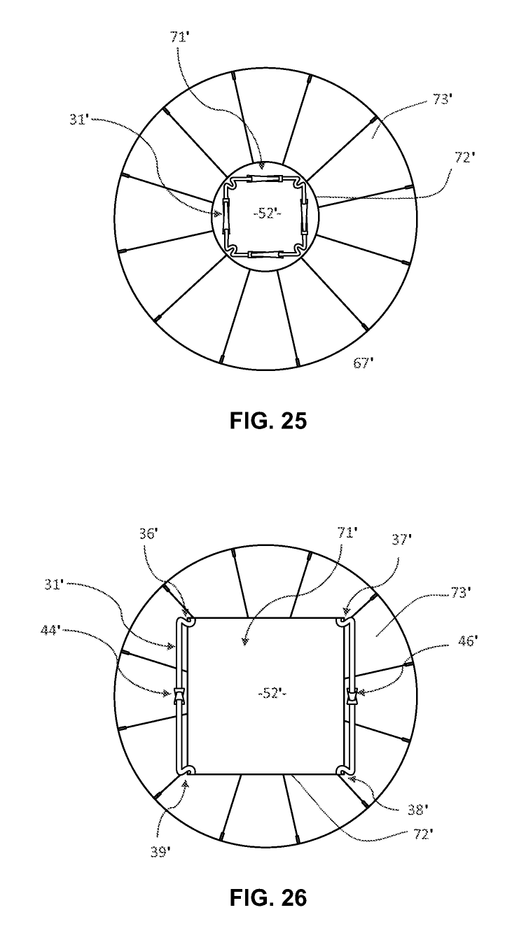

FIG. 25 refers to the usage of the invention, is an illustration of the placement of the device 31′ of FIG. 17, within the non-dilated pupil 71′ of the eye. A forceps (not shown here) carries the device or an injector (not shown here) delivers the device through an incision in the cornea into the anterior chamber of the eye. The flexible square ring is held with a forceps and the notches are engaged to the pupil margin 72′.

FIG. 26 is an illustration of usage of the invention in further detail, and shows the device 31′ in an expanded state and engaged to the pupil margin, resulting in an expanded pupil. The device 31′ is engaged to the pupil margin 72′ by notches 36′, 37′, 38′ and 39′. Supporting parts 40′ and 42′ are pushed apart using two Kuglen hooks (not shown here) to expand supporting parts 41′ and 43′. Supporting parts 40′ and 42′ are tucked under the pupil margin 72′. Supporting parts 41′ and 43′, lying anterior to the pupil margin are also pushed apart to expand the supporting parts 40′ and 42′. Expansion of the enclosed space 52′ results in expansion of the pupil 71′. The pupil is expanded to the desired size. Supporting parts 40′ and 42′ are tucked under the pupil margin 72′ and iris 73′ and are not visible. Supporting parts 41′ and 43′ and joints 44′ and 46′ lying anterior to the iris 73′ are visible.

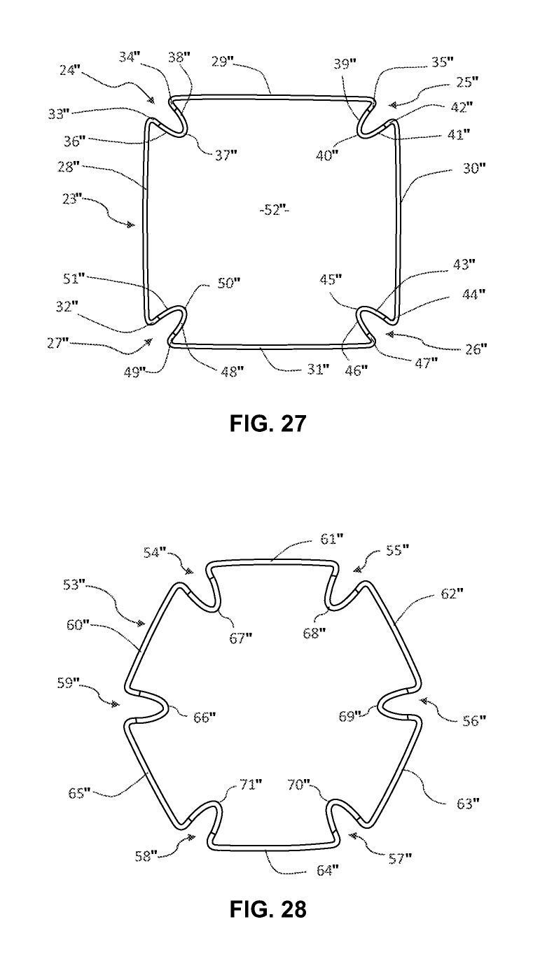

FIG. 27 is an enlarged diagrammatic top perspective of a square closed ring 23″ of the present invention, in its expanded undeformed state showing shape memory resilient notches 24″, 25″, 26″ and 27″ and shape retaining pliant flanges 28″, 29″, 30″ and 31″. Flange 28″ has a first end 32″ and a second end 33″. Flange 29″ has a first end 34″ and a second end 35″. Notch 24″ has a first limb 36″, which connects the second end 33″ of flange 28″ to the blind end 37″ of the notch. Second limb 38″ of notch 24″ connects the blind end 37″ to the first end 34″ of flange 29″. Limb 39″ of notch 25″, connects second end 35″ of flange 29″ to blind end 40″. Limb 41″ connects blind end 40″ of notch 25″ to first end 42″ of flange 30″. Limb 43″ of notch 26″, connects second end 44″ of flange 30″ to blind end 45″. Limb 46″ connects blind end 45″ of notch 26″ to first end 47″ of flange 31″. Limb 48″ of notch 27″, connects second end 49″ of flange 31″ to blind end 50″. Limb 51″ connects blind end 50″ of notch 27″ to first end 32″ of flange 28″. In the enlarged and undeformed state, the ring 23″ encloses a space 52″. Notches 24″, 25″, 26″ and 27″ receive and engage different parts of the pupil margin. In one method of engagement, the pupil margin and adjacent iris pass over limb 51″, flange 28″, limb 36″, limb 41″, flange 30″ and limb 43″ and under limb 38″, flange 29″, limb 39″, limb 46″, flange 31″ and limb 48″. In an alternate method of engagement, the pupil margin and adjacent iris pass under limb 51″, flange 28″, limb 36″, limb 41″, flange 30″ and limb 43″ and over limb 38″, flange 29″, limb 39″, limb 46″, flange 31″ and limb 48″. The enclosed square space, with blind ends 37″, 40″, 45″ and 50″ as the corners, represents the expanded pupil and provides visualization and access to structures lying deeper to this plane.

FIG. 28 is an enlarged diagrammatic top perspective of a hexagonal closed ring 53″ of the present invention, in its expanded undeformed state showing shape memory resilient notches 54″, 55″, 56″, 57″, 58″ and 59″ and shape retaining pliant flanges 60″, 61″, 62″, 63″, 64″ and 65″ and enclosing a space 66″. Notches 54″, 55″, 56″, 57″, 58″ and 59″ receive and engage different parts of the pupil margin. In one method of engagement, the pupil margin and adjacent iris pass over flanges 60″, 62″, and 64″ and under flanges 61″, 63″ and 65″. In an alternate method of engagement, the pupil margin and adjacent iris pass under flanges 60″, 62″, and 64″ and over flanges 61″, 63″ and 65″. Blind ends 66″, 67″, 68″, 69″, 70″ and 71″ of notches form the six corners of an enclosed hexagonal space, which represents the expanded pupil and provides visualization and access to structures lying deeper to this plane.

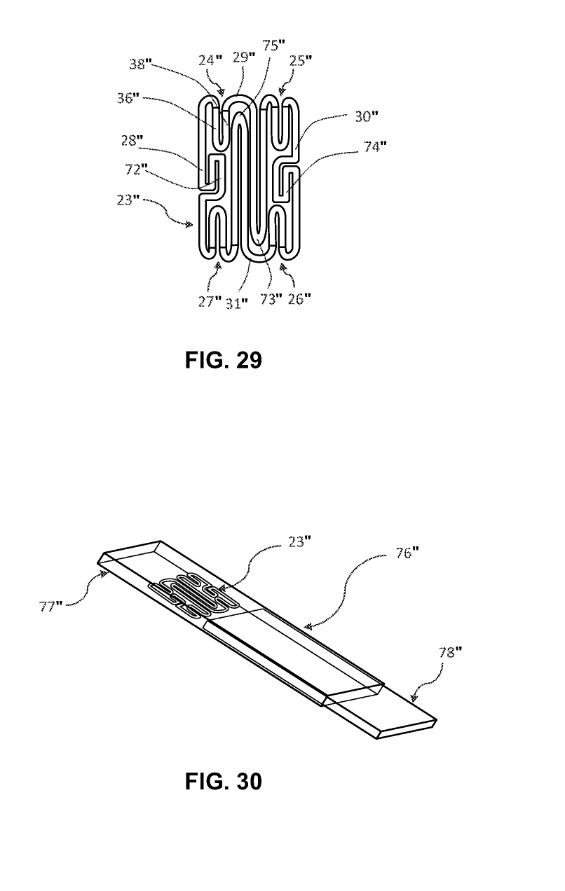

FIG. 29 is an enlarged diagrammatic top view of a contracted state of the device of FIG. 27, showing all four notches 24″, 25″, 26″, and 27″ and all four flanges 28″, 29″, 30″ and 31″, in a deformed state. Limbs 36″ and 38″ of notch 24″ are approximated to each other and the notch is narrowed. Similarly, notches 25″, 26″ and 27″ are narrowed. Flange 28″ is folded to form loop 72″. Flange 29″ is folded to form loop 73″. Flange 30″ is folded over itself to form loop 74″. Flange 31″ is folded over itself to form loop 75″. Such folding of the notches and flanges results in obliteration of the enclosed space 52″.

FIG. 30 is an enlarged diagrammatic top perspective view of a contracted state of the device 23″ of FIG. 27, showing it compactly nested in a delivery device 76″, with all four notches 24″, 25″, 26″, and 27″ and all four flanges 28″, 29″, 30″ and 31″ deformed. The delivery device 76″ comprises an outer tube 77″ and a plunger 78″. Outer tube 77″ carries the device 23″ through a corneal incision into the eye. Forward motion of the plunger 78″ expels the device 23″ into the anterior chamber of the eye.

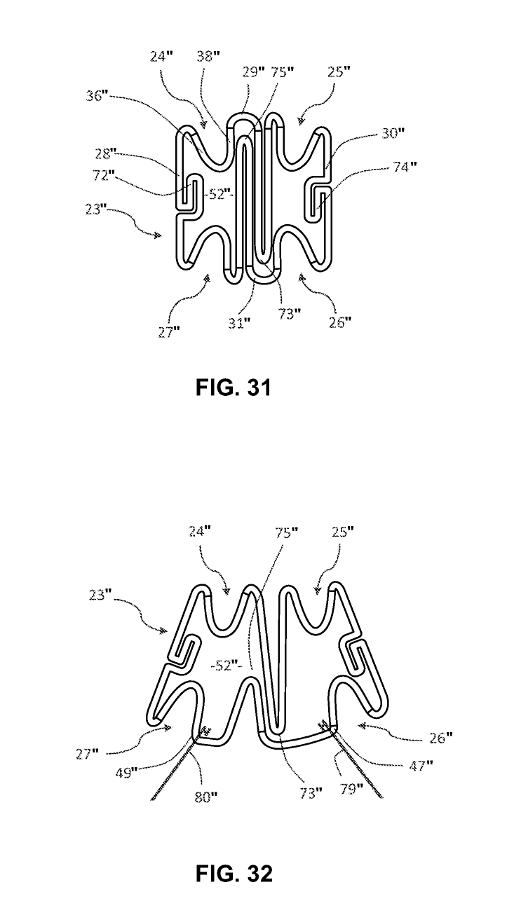

FIG. 31 is an enlarged diagrammatic top view of a contracted state of the device 23″ of FIG. 27, after expulsion from the delivery device into the eye, showing all four notches 24″, 25″, 26″, and 27″ undeformed and all four flanges 28″, 29″, 30″ and 31″ still deformed. The resilient limbs 36″ and 38″ of notch 24″, having shape memory, have moved apart and the notch has returned to its undeformed shape. Similarly, notches 25″, 26″, and 27″ have also returned to their undeformed states. The pliant flanges 28″, 29″, 30″ and 31″ being shape retaining, remain deformed. This contracted state of the device allows the notches to engage the margin of the non-dilating pupil without any stretching.

FIG. 32 is an enlarged diagrammatic top view of the device of FIG. 27, showing the contracted ring 23″, being enlarged manually using two Kuglen hooks 79″ and 80″. One Kuglen hook 79″ engages the first end 47″ of flange 31″, and a second Kuglen hook 80″ engages the second end 49″ of flange 31″. As the two Kuglen hooks are moved apart, the loop 75″ of shape retaining pliant flange 31″ gradually straightens in a very controlled manner. Similarly, loops 72″, 73″ and 74″, are straightened one by one to produce controlled expansion of the device and enlargement of the enclosed space 52″ and expansion of the pupil. As the device is expanded, the pupil margin and adjacent iris passes either over or under opposite flanges.

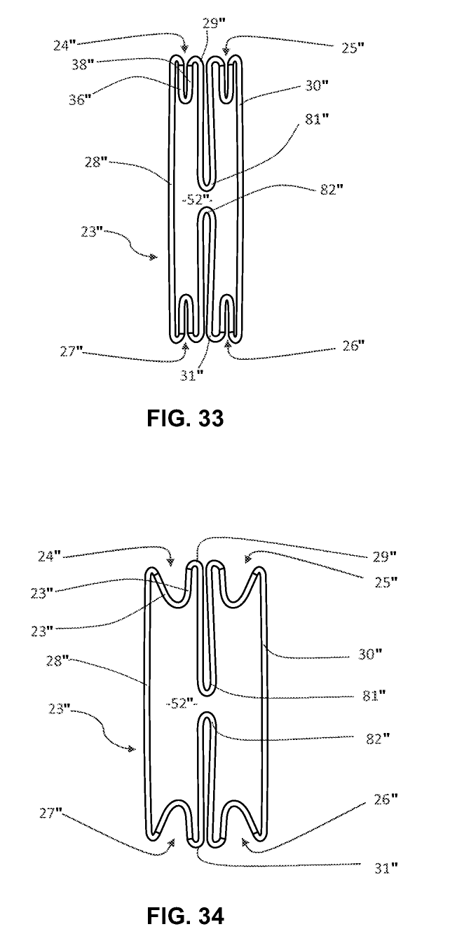

FIG. 33 is an enlarged diagrammatic top view of a contracted state of the device of FIG. 27, showing all four notches 24″, 25″, 26″, and 27″ and only two flanges 29″ and 31″ deformed. Limbs 36″ and 38″ of notch 24″ are approximated to each other and the notch is narrowed. Similarly, notches 25″, 26″ and 27″ are narrowed. Flange 29″ is folded to form loop 81″ and flange 31″ is folded to form loop 82″. Flanges 28″ and 30″ remain undeformed. This results in contraction of the device breadth wise to facilitate easy insertion through a small incision. This contracted state also facilitates compact nesting within a delivery device.

FIG. 34 is an enlarged diagrammatic top view of a contracted state of the device of FIG. 27, after expulsion from a delivery device, after having been in a contracted state of FIG. 33, showing all four notches 24″, 25″, 26″, and 27″ undeformed and two flanges 29″ and 31″ still deformed. The device is merely placed in the anterior chamber of the eye and the notches do not receive the pupil margin at this stage.

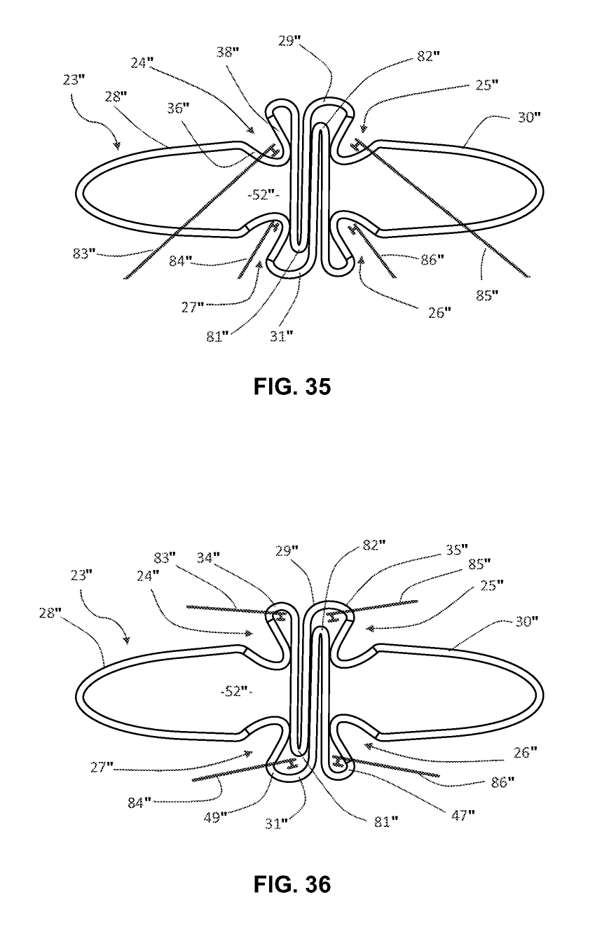

FIG. 35 is an enlarged diagrammatic top view of a contracted state of the device of FIG. 27, showing deformation of the two previously undeformed flanges 28″ and 30″ of FIG. 34. Kuglen hooks 83″ and 84″ engage notches 24″ and 27″ respectively and draw them towards each other causing flange 28″ to be deformed. Kuglen hooks 85″ and 86″ engage notches 25″ and 26″ respectively and draw them towards each other causing flange 30″ to be deformed. This brings the four notches 24″, 25″, 26″, and 27″ close to each other and enables the notches receive the non dilating pupil margin without any stretching. As flanges 28″ and 30″ are deformed they remain over the iris.

FIG. 36 is an enlarged diagrammatic top view of a contracted state of the device of FIG. 27, showing deformation of flanges 28″ and 30″ as in FIG. 35. Notches 24″, 25″, 26″, and 27″ receive the pupil margin in a manner that the pupil margin and adjacent iris pass under flanges 28″ and 30″, over first end 34″ and second end 35″ of flange 29″, and over first end 47″ and second end 49″ of flange 31″. Kuglen hook 83″ now engages the first end 34″ of flange 29″ and Kuglen hook 85″ now engages the second end 35″ of flange 29″ to straighten the loop 81″ of flange 29″. Similarly, Kuglen hook 86″ now engages the first end 47″ of flange 31″ and Kuglen hook 84″ now engages the second end 49″ of flange 31″ to straighten the loop 82″ of flange 31″. The loops 81″ and 82″ are straightened in a manner that the pupil margin and adjacent iris pass over the straightened flanges 29″ and 31″ and under the straightened flanges 28″ and 30″. This causes expansion of the device and in effect expansion of the pupil.

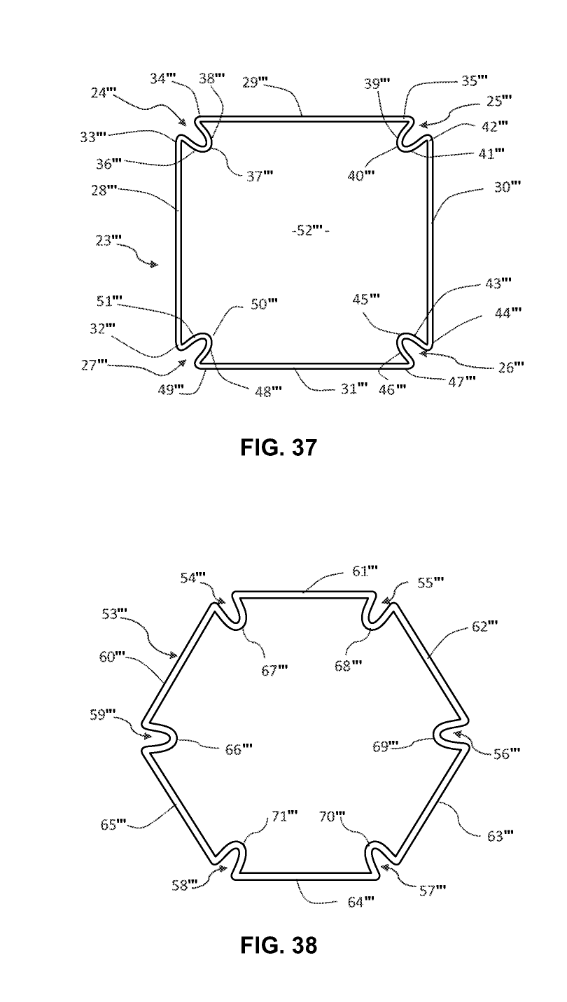

FIG. 37 is an enlarged diagrammatic top perspective of a square closed ring 23′″ of the present invention made of shape memory material, in its expanded state showing notches 24′″, 25′″, 26′″ and 27′″ and flanges 28′″, 30′″ and 31′″. Flange 28′″ has a first end 32′″ and a second end 33′″. Flange 29′″ has a first end 34′″ and a second end 35′″. Notch 24′″ has a first limb 36′″, which connects the second end 33′″ of flange 28′″ to the blind end 37′″ of the notch. Second limb 38′″ of notch 24′″ connects the blind end 37′″ to the first end 34′″ of flange 29′″. Limb 39′″ of notch 25′″, connects second end 35′″ of flange 29′″ to blind end 40′″. Limb 41′″ connects blind end 40′″ of notch 25′″ to first end 42′″ of flange 30′″. Limb 43′″ of notch 26′″, connects second end 44′″ of flange 30′″ to blind end 45′″. Limb 46′″ connects blind end 45′″ of notch 26′″ to first end 47′″ of flange 31′″. Limb 48′″ of notch 27′″, connects second end 49′″ of flange 31′″ to blind end 50′″. Limb 51′″ connects blind end 50′″ of notch 27′″ to first end 32′″ of flange 28′″. In the enlarged and undeformed state, the ring 23′″ encloses a space 52′″. Notches 24′″, 25′″, 26′″ and 27′″ receive and engage different parts of the pupil margin. In one method of engagement, the pupil margin and adjacent iris pass over limb 51′″, flange 28′″, limb 36′″, limb 41′″, flange 30′″ and limb 43′″ and under limb 38′″, flange 29′″, limb 39′″, limb 46′″, flange 31′″ and limb 48′″. In an alternate method of engagement, the pupil margin and adjacent iris pass under limb 51′″, flange 28′″, limb 36′″, limb 41′″, flange 30′″ and limb 43′″ and over limb 38′″, flange 29′″, limb 39′″, limb 46′″, flange 31′″ and limb 48′″. The enclosed square space, with blind ends 37′″, 45′″ and 50′″ as the corners, represents the expanded pupil and provides visualization and access to structures lying deeper to this plane.

FIG. 38 is an enlarged diagrammatic top perspective of a hexagonal closed ring 53′″ of the present invention made of shape memory material, in its expanded state, showing notches 54′″, 55′″, 56′″, 57′″, 58′″ and 59′″ and flanges 60′″, 61′″, 62′″, 63′″, 64′″ and 65′″ and enclosing a space 66′″. Notches 54′″, 55′″, 56′″, 57′″, 58′″ and 59′″ receive and engage different parts of the pupil margin. In one method of engagement, the pupil margin and adjacent iris pass over flanges 60′″, 62′″, and 64′″ and under flanges 61′″, 63′″ and 65′″. In an alternate method of engagement, the pupil margin and adjacent iris pass under flanges 60′″, 62′″, and 64′″ and over flanges 61′″, 63′″ and 65′″. Blind ends 66′″, 67′″, 68′″, 69′″, 70′″ and 71′″ of notches form the six corners of an enclosed hexagonal space, which represents the expanded pupil and provides visualization and access to structures lying deeper to this plane.

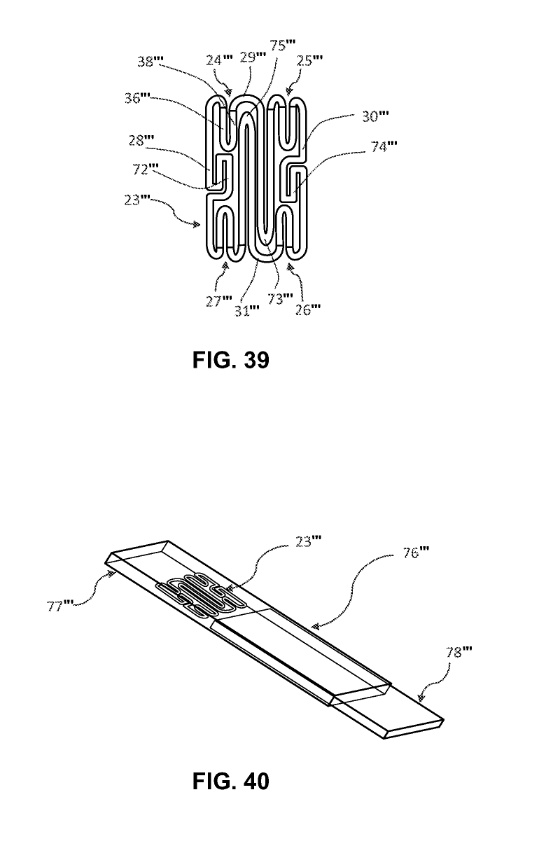

FIG. 39 is an enlarged diagrammatic top view of a contracted state of the device of FIG. 37, showing all four notches 24′″, 25′″, 26″, and 27′″ and all four flanges 28′″, 29′″, 30′″ and 31′″, in a deformed state. Limbs 36′″ and 38′″ of notch 24′″ are approximated to each other and the notch is narrowed. Similarly, notches 25′″, 26′″ and 27′″ are narrowed. Flange 28′″ is folded to form loop 72′″. Flange 29′″ is folded to form loop 73′″. Flange 30′″ is folded over itself to form loop 74′″. Flange 31′″ is folded over itself to form loop 75′″. Such folding of the notches and flanges results in obliteration of the enclosed space 52′″.

FIG. 40 is an enlarged diagrammatic top perspective view of a contracted state of the device 23′″ of FIG. 39, showing it compactly nested in a delivery device 76′″, with all four notches 24′″, 25′″, 26′″, and 27′″ and all four flanges 28′″, 29′″, 30′″ and 31′″ deformed. The delivery device 76′″ comprises an outer tube 77′″ and a plunger 78′″. Outer tube 77′″ carries the device 23′″ through a corneal incision into the eye. Forward motion of the plunger 78′″ expels the device 23′″ into the anterior chamber of the eye.

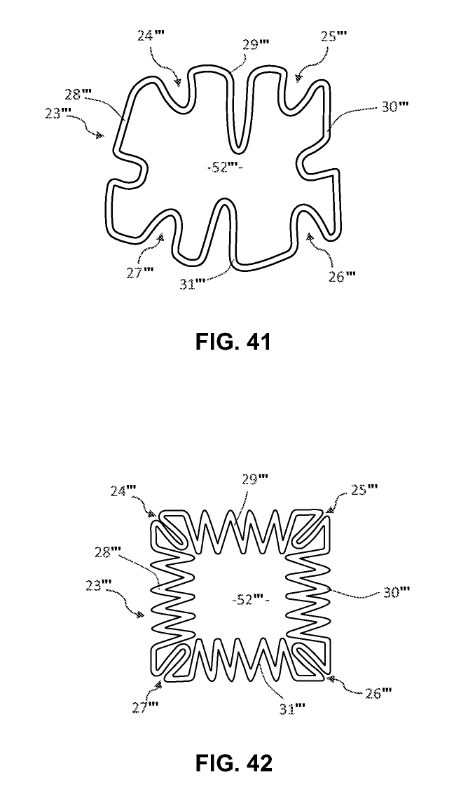

FIG. 41 is an enlarged diagrammatic top view of a partially expanded state of the device 23′″ of FIG. 39, after expulsion from the delivery device into the eye, showing all four notches 24′″, 25′″, 26′″, and 27′″ and all four flanges 28′″, 29′″, 30′″ and 31′″ having partially regained their shape due to shape memory. This partially expanded state of the device allows the notches to be engaged to the margin of the non-dilating pupil without any stretching. The device self-expands by moving from the shape of FIG. 39 to the shape of FIG. 37 due to shape memory.

FIG. 42 is an enlarged diagrammatic top view of another embodiment of the contracted state of the device of FIG. 37, showing the notches and flanges of ring 23′″ folded to result in shape that is smaller in size. Notches 24′″, 25′″, 26′″ and 27′″ are narrowed and flanges 28′″, 29′″, 30′″ and 31′″ are folded in a zigzag manner. The central space 52′″ is much smaller in this contracted state.

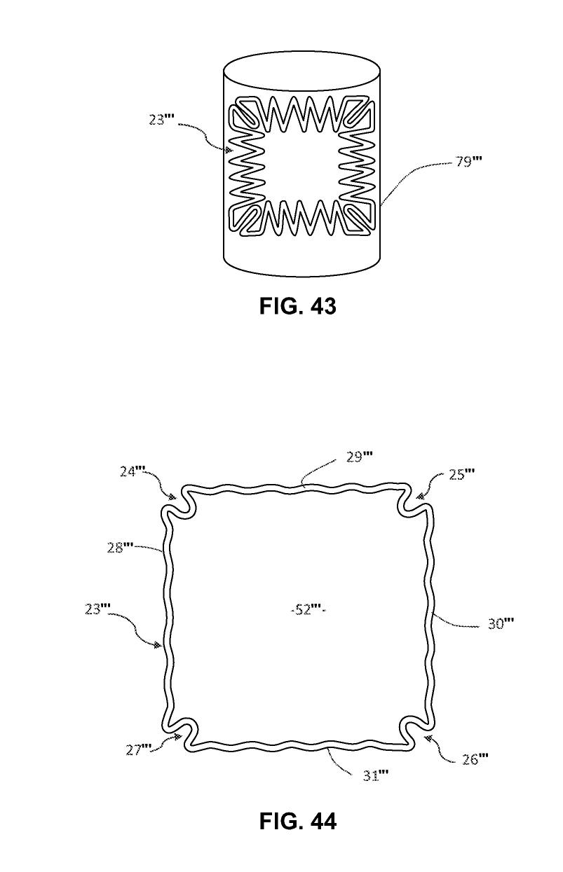

FIG. 43 is an enlarged diagrammatic top view of a contracted state of the device 23′″ of FIG. 42, showing it compactly nested within a sleeve 79′″, with all four notches and flanges folded. The sleeve 79′″ carries the device 23′″ through a corneal incision into the eye.

FIG. 44 is an enlarged diagrammatic top view of the device of FIG. 42, in nearly fully self expanded state showing the four notches 24′″, 25′″, 26′″, and 27′″ fully expanded and the flanges 28′″, 29′″, 30′″ and 31′″ nearly straightened. The central space 52′″ is also expanded. The device self-expands by moving from the shape of FIG. 42 to the shape of FIG. 37 due to shape memory.

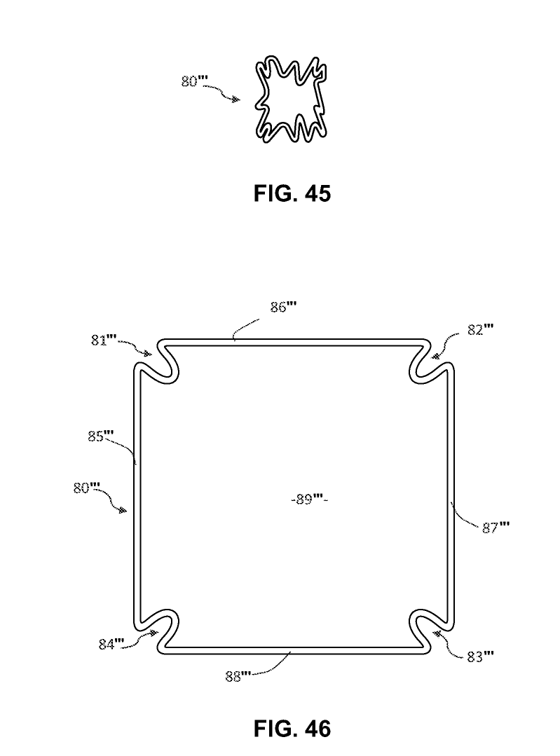

FIG. 45 is an enlarged diagrammatic top view of a contracted state of a device of the present invention made of shape memory self-enlarging material. The device 80′″ is a small closed ring formed from a tortuous strand. The notches and flanges are not discernible due to the tortuosity of the strand.

FIG. 46 is an enlarged diagrammatic top view of a fully expanded state of the device of FIG. 45, showing notches 81′″, 82′″, 83′″, and 84′″ and straightened flanges 85′″, 86′″, 87′″ and 88′″. The device encloses a space 89′″. The circumference of the device of FIG. 46 is larger than that of the device of FIG. 45.

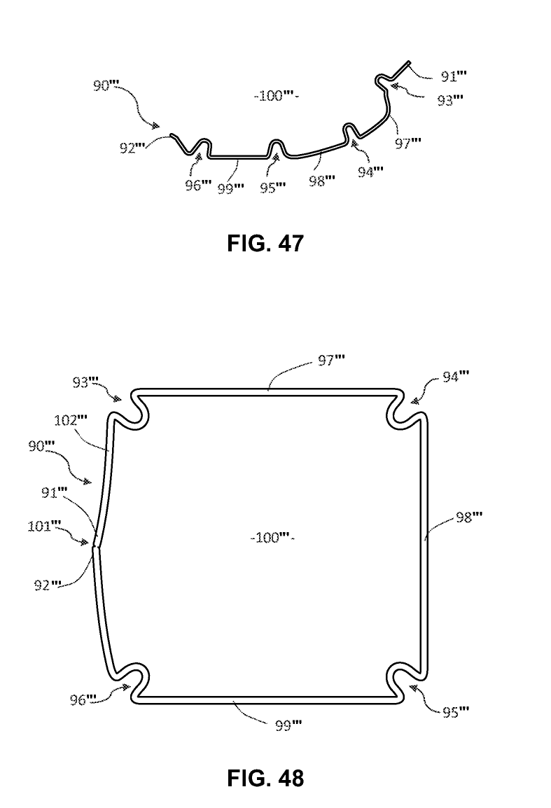

FIG. 47 is an enlarged diagrammatic top view of a contracted and open ring state of a device of the present invention made of self-enlarging, self-healing and self-reconfiguring programmable material showing device 90′″ with free ends 91′″ and 92′″. Sides 97′″, 98′″ and 99′″, connect notches 93′″, 94′″, 95′″ and 96′″. The device 90′″ partially encloses a space 100′″.

FIG. 48 is an enlarged diagrammatic top view of a fully expanded and closed ring state of a device of the present invention formed by self-enlargement, self-healing and self-reconfiguration of the device 90′″ of FIG. 47, showing the free ends 91′″ and 92′″ coming together in a programmed manner and joining by self healing at joint 101′″ to form flange 102′″. The device completely encloses the space 100. Notches 93′″, 94′″, 95′″ and 96′″ and flanges 97′″, 98′″ and 99′″ of the device of FIG. 14 are larger than the corresponding notches and flanges of FIG. 47.

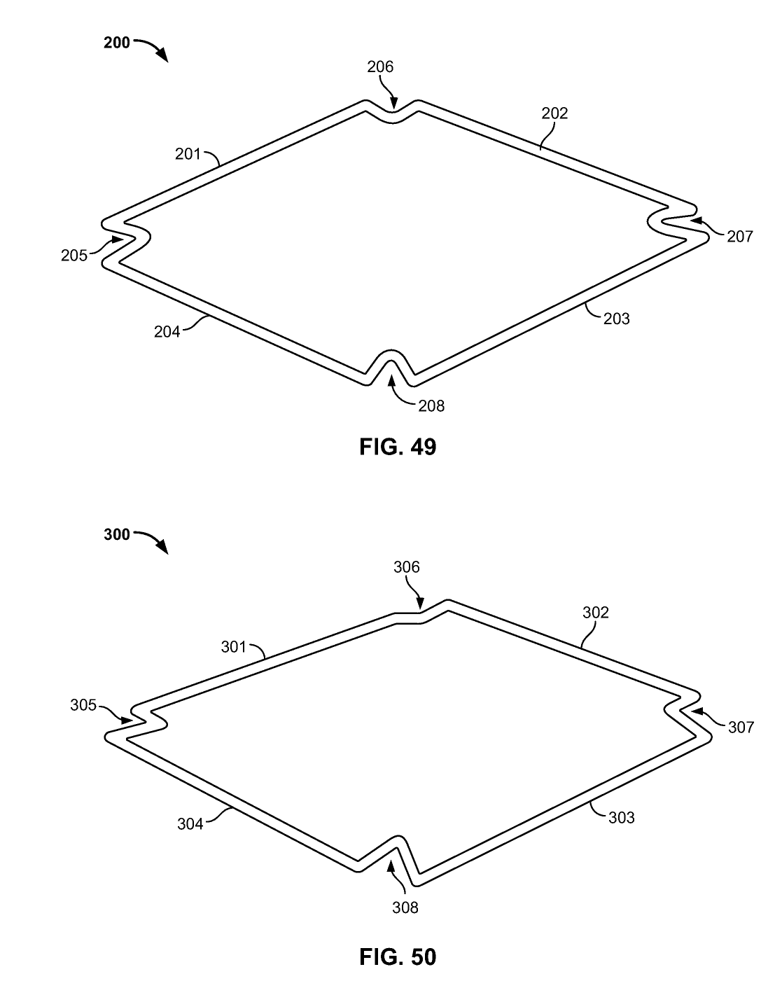

FIG. 49 is an enlarged perspective view of a device 200 having four flanges 201, 202, 203 and 204, all of which define a first plane. The four flanges 201, 202, 203 and 204 define four blind corners 205, 206, 207 and 208, as illustrated in FIG. 49. All of the blind corners 205, 206, 207 and 208 define a second plane 0.1 to 0.5 mm above the first plane defined by the flanges 201, 202, 203 and 204. Though not contained entirely in a single plane, the device 200 does not snag the incision because the notches defined by the blind corners 205, 206, 207 and 208 can completely straighten out as the device 200 negotiates the incision. The advantage of this embodiment is that the device conforms to the anterior convexity of the crystalline lens and iris.

FIG. 50 is an enlarged perspective view of a device 300 having four flanges 301, 302, 303 and 304. Flanges 301 and 303 define a first plane, while flanges 302 and 304 define a second plane, e.g., 0.1 to 0.5 mm above the first plane. The four flanges 301, 302, 303 and 304 define four blind corners 305, 306, 307 and 308, as illustrated in FIG. 50. The blind corners 305, 306, 307 and 308 lie at either the first plane or second plane, or in between these planes. Though not entirely in a single plane, the device 300 does not snag the incision because the notches defined by the blind corners 305, 306, 307 and 308 can completely straighten out as the device negotiates the incision. The advantages of this embodiment are that alternate flanges are at a lower plane and are easily tucked under the pupil margin and there is less bending of the iris.

In an alternate embodiment, the device may be employed as a long-term and/or permanent prosthetic. More particularly, the device may be placed in the eye as a prosthetic to achieve long-term pupil dilation.

Although different ways of deforming the device have been described and illustrated, it is understood that the device may be deformed in other ways to serve the purpose. Although a square shaped configuration is described, it is understood that the present invention may have any polygonal shape, including a rectangular shape and a hexagonal shape. Although a closed ring configuration is described, it is understood that the present invention may be a closed ring, which can be converted to an open ring. It is also understood that the device may have an open ring configuration or an open ring configuration which can be converted to a closed ring. It is understood that the present invention may be placed in the eye using a forceps, Kuglen hook, or a delivery device like an Injector, Shooter or a Carrier, with or without the use of a cartridge.

Those ordinarily skilled in the art can make changes in the embodiments described and illustrated, without altering the concepts of the present invention. Hence, it is to be understood that the invention is not limited to the descriptions, illustrations and examples, but includes all modifications within the scope of this invention.

1. A device for enlarging a pupil of an eye during surgery, the eye including an iris and the pupil including a pupillary margin adjacent the iris, the device comprising:
an expandable continuous ring enclosing a central space and having at least four slidably interconnected units made from a resiliently flexible material, including
a first unit having a first leg with a first end, a second leg with a second end, and a first corner portion connectively disposed between said first leg and said second leg and forming first receiving means for receiving a first portion of the pupillary margin, said first end including first engaging means and said second end including second engaging means;
a second unit having a third leg with a third end, a fourth leg with a fourth end, and a second corner portion connectively disposed between said third leg and said fourth leg and forming second receiving means for receiving a second portion of the pupillary margin, said third end including third engaging means and said fourth end including fourth engaging means;
a third unit having a fifth leg with a fifth end, a sixth leg with a sixth end, and a third corner portion connectively disposed between said fifth leg and said sixth leg and forming third receiving means for receiving a third portion of the pupillary margin, said fifth end including fifth engaging means and said sixth end including sixth engaging means; and
a fourth unit having a seventh leg with a seventh end, an eighth leg with an eighth end, and a fourth corner portion connectively disposed between said seventh leg and said eighth leg and forming fourth receiving means for receiving a fourth portion of the pupillary margin, said seventh end including seventh engaging means and said eighth end including eighth engaging means,
said first corner portion, said second corner portion, said third corner portion and said fourth corner portion extending into said central space;
wherein said second engaging means slidably engages said third end and said third engaging means slidably engages said second end to form a first adjustable flange between said first corner portion and said second corner portion;
wherein said fourth engaging means slidably engages said fifth end and said fifth engaging means slidably engages said fourth end to form a second adjustable flange between said second corner portion and said third corner portion;
wherein said sixth engaging means slidably engages said seventh end and said seventh engaging means slidably engages said sixth end to form a third adjustable flange between said third corner portion and said fourth corner portion; and
wherein said eighth engaging means slidably engages said first end and said first engaging means slidably engages said eighth end to form a fourth adjustable flange between said fourth corner portion and said first corner portion;
said first, second, third and fourth adjustable flanges defining a plane and forming said continuous ring, said continuous ring being moveable between a contracted position and an expanded position, wherein in said contracted position, said first end is proximate said fourth corner portion and distal said eighth end, said eighth end is proximate said first corner portion, said second end is proximate said second corner portion and distal said third end, said third end is proximate said first corner portion, said fourth end is proximate said third corner portion and distal said fifth end, said fifth end is proximate said second corner portion, said sixth end is proximate said fourth corner portion and distal said seventh end, and said seventh end is proximate said third corner portion, such that said central space has a first area, and
wherein in said expanded position, said first end is distal said fourth corner portion and proximate said eighth end, said eighth end is distal said first corner portion, said second end is distal said second corner portion and proximate said third end, said third end is distal said first corner portion, said fourth end is distal said third corner portion and proximate said fifth end, said fifth end is distal said second corner portion, said sixth end is distal said fourth corner portion and proximate said seventh end, and said seventh end is distal said third corner portion, such that said central space has a second area greater than said first area.

2. The device of claim 1, said first corner portion including a first blind end, a first limb connecting said fourth flange to said first blind end on one side thereof, and a second limb connecting said first flange to said first blind end on an opposite side thereof so as to form said first receiving means, and wherein said first receiving means includes a first notch sized and shaped to receive the first portion of the pupillary margin such that the iris passes over said fourth flange and said first limb and under said second limb and said first flange;
said second corner portion including a second blind end, a third limb connecting said first flange to said second blind end on one side thereof, and a fourth limb connecting said second flange to said second blind end on an opposite side thereof so as to form said second receiving means, and wherein said second receiving means includes a second notch sized and shaped to receive the second portion of the pupillary margin such that the iris passes under said first flange and said third limb and over said fourth limb and said second flange;
said third corner portion including a third blind end, a fifth limb connecting said second flange to said third blind end on one side thereof, and a sixth limb connecting said third flange to said third blind end on an opposite side thereof so as to form said third receiving means, and wherein said third receiving means includes a third notch sized and shaped to receive the third portion of the pupillary margin such that the iris passes over said second flange and said fifth limb and under said sixth limb and said third flange; and
said fourth corner portion including a fourth blind end, a seventh limb connecting said third flange to said fourth blind end on one side thereof, and an eighth limb connecting said fourth flange to said fourth blind end on an opposite side thereof so as to form said fourth receiving means, and wherein said fourth receiving means includes a fourth notch sized and shaped to receive the fourth portion of the pupillary margin such that the iris passes under said third flange and said seventh limb and over said eighth limb and said fourth flange.

3. The device of claim 1, wherein said first notch, said second notch, said third notch and said fourth notch are contained within said plane.

4. The device of claim 1, wherein said first engaging means includes a first hole being sized and shaped to slidably receive said eighth end therein, said second engaging means includes a second hole being sized and shaped to slidably receive said third end therein, said third engaging means includes a third hole being sized and shaped to slidably receive said second end therein, said fourth engaging means includes a fourth hole being sized and shaped to slidably receive said fifth end therein, said fifth engaging means includes a fifth hole being sized and shaped to slidably receive said fourth end therein, said sixth engaging means includes a sixth hole being sized and shaped to slidably receive said seventh end therein, said seventh engaging means includes a seventh hole being sized and shaped to slidably receive said sixth end therein, and said eighth engaging means includes an eighth hole being sized and shaped to slidably receive said first end therein.

5. The device of claim 4, wherein said first leg has a substantially circular cross-sectional area proximate said first corner portion, and a substantially rectangular cross-sectional area proximate said first end;
wherein said second leg has a substantially circular cross-sectional area proximate said first corner portion, and a substantially rectangular cross-sectional area proximate said second end;
wherein said third leg has a substantially circular cross-sectional area proximate said second corner portion, and a substantially rectangular cross-sectional area proximate said third end;
wherein said fourth leg has a substantially circular cross-sectional area proximate said second corner portion, and a substantially rectangular cross-sectional area proximate said fourth end;
wherein said fifth leg has a substantially circular cross-sectional area proximate said third corner portion, and a substantially rectangular cross-sectional area proximate said fifth end;
wherein said sixth leg has a substantially circular cross-sectional area proximate said third corner portion, and a substantially rectangular cross-sectional area proximate said sixth end;
wherein said seventh leg has a substantially circular cross-sectional area proximate said fourth corner portion, and a substantially rectangular cross-sectional area proximate said seventh end; and
wherein said eighth leg has a substantially circular cross-sectional area proximate said fourth corner portion, and a substantially rectangular cross-sectional area proximate said eighth end.

6. The device of claim 1, wherein said first engaging means and said eighth engaging means cooperate to include a first telescopic slip joint formed by said first end and said eighth end, said eighth end having a first diameter and said first end having a second diameter larger than said first diameter, whereby said eighth end may be slidably received within said first end;
wherein said second engaging means and said third engaging means cooperate to include a second telescopic slip joint formed by said second end and said third end, said second end having a third diameter and said third end having a fourth diameter larger than said third diameter, whereby said second end may be slidably received within said third end;
wherein said fourth engaging means and said fifth engaging means cooperate to include a third telescopic slip joint formed by said fourth end and said fifth end, said fourth end having a fifth diameter and said fifth end having a sixth diameter larger than said fifth diameter, whereby said fourth end may be slidably received within said fifth end; and
wherein said sixth engaging means and said seventh engaging means cooperate to include a fourth telescopic slip joint formed by said sixth end and said seventh end, said sixth end having a seventh diameter and said seventh end having an eighth diameter larger than said seventh diameter, whereby said sixth end may be slidably received within said seventh end.

7. The device of claim 1, wherein said ring is moveable between an undeformed state and a deformed state.

8. The device of claim 7, wherein said ring is manually moveable from its said undeformed state to its said deformed state, and wherein said ring automatically returns to its said undeformed state from its said deformed state.

9. A device for enlarging a pupil of an eye during surgery, the eye including an iris and the pupil including a pupillary margin adjacent the iris, the device comprising:
a ring at least partially enclosing a central space and having at least three flanges defining a plane, including a first flange, a second flange and a third flange, a first corner portion connectively disposed between said first flange and said second flange and forming first receiving means for receiving a first portion of the pupillary margin, and a second corner portion connectively disposed between said second flange and said third flange and forming second receiving means for receiving a second portion of the pupillary margin, said first corner portion and said second corner portion extending into said central space,
said first corner portion including a first blind end, a first limb connecting said first flange to said first blind end on one side thereof, and a second limb connecting said second flange to said first blind end on an opposite side thereof so as to form said first receiving means, and wherein said first receiving means includes a first notch sized and shaped to receive the first portion of the pupillary margin such that the iris passes over said first flange and said first limb and under said second limb and said second flange,
said second corner portion including a second blind end, a third limb connecting said second flange to said second blind end on one side thereof, and a fourth limb connecting said third flange to said second blind end on an opposite side thereof so as to form said second receiving means, and wherein said second receiving means includes a second notch sized and shaped to receive the second portion of the pupillary margin such that the iris passes under said second flange and said third limb and over said fourth limb and said third flange, and
wherein at least one of said least three flanges has an adjustable length.

10. The device of claim 9, wherein each of said at least three flanges has an adjustable length.

11. The device of claim 9, wherein said ring is made from a resiliently flexible strand such that said ring is moveable between an undeformed state and a deformed state.

12. The device of claim 11, wherein said ring is manually moveable from its said undeformed state to its said deformed state, and wherein said ring automatically returns to its said undeformed state from its said deformed state.

13. The device of claim 9, wherein said adjustable length is slidably adjustable.

14. The device of claim 13, wherein said slidably adjustable length is adjustable telescopically.

15. The device of claim 9, wherein said ring is closed.

16. A device for enlarging a pupil of an eye during surgery, the eye including an iris and the pupil including a pupillary margin adjacent the iris, the device comprising:
a ring at least partially enclosing a central space and having at least three flanges defining a plane, including a first flange, a second flange and a third flange, a first corner portion connectively disposed between said first flange and said second flange and forming first receiving means for receiving a first portion of the pupillary margin, and a second corner portion connectively disposed between said second flange and said third flange and forming second receiving means for receiving a second portion of the pupillary margin, said first corner portion and said second corner portion extending into said central space,
said first corner portion including a first blind end, a first limb connecting said first flange to said first blind end on one side thereof, and a second limb connecting said second flange to said first blind end on an opposite side thereof so as to form said first receiving means, and wherein said first receiving means includes a first notch sized and shaped to receive the first portion of the pupillary margin such that the iris passes over said first flange and said first limb and under said second limb and said second flange,
said second corner portion including a second blind end, a third limb connecting said second flange to said second blind end on one side thereof, and a fourth limb connecting said third flange to said second blind end on an opposite side thereof so as to form said second receiving means, and wherein said second receiving means includes a second notch sized and shaped to receive the second portion of the pupillary margin such that the iris passes under said second flange and said third limb and over said fourth limb and said third flange, and
wherein at least one of said first notch and said second notch is formed from a shape memory resilient material, and wherein at least one of said at least three flanges is formed from a shape retaining pliant material, such that said ring is moveable between an undeformed state and a deformed state.

17. The device of claim 16, wherein said ring is manually moveable from its said undeformed state to its said deformed state, and wherein said ring is at least partially manually moveable from its said deformed state to its said undeformed state.

18. The device of claim 17, wherein said at least one of said at least three flanges formed from a shape retaining pliant material is manually moveable between an undeformed flange state and a deformed flange state, and wherein said at least one of said first notch and said second notch formed from a shape memory resilient material is automatically moveable from a deformed notch state to an undeformed notch state.

19. A device for enlarging a pupil of an eye during surgery, the eye including an iris and the pupil including a pupillary margin adjacent the iris, the device comprising:
a ring at least partially enclosing a central space and having at least three flanges defining a plane, including a first flange, a second flange and a third flange, a first corner portion connectively disposed between said first flange and said second flange and forming first receiving means for receiving a first portion of the pupillary margin, and a second corner portion connectively disposed between said second flange and said third flange and forming second receiving means for receiving a second portion of the pupillary margin, said first corner portion and said second corner portion extending into said central space,
said first corner portion including a first blind end, a first limb connecting said first flange to said first blind end on one side thereof, and a second limb connecting said second flange to said first blind end on an opposite side thereof so as to form said first receiving means, and wherein said first receiving means includes a first notch sized and shaped to receive the first portion of the pupillary margin such that the iris passes over said first flange and said first limb and under said second limb and said second flange,
said second corner portion including a second blind end, a third limb connecting said second flange to said second blind end on one side thereof, and a fourth limb connecting said third flange to said second blind end on an opposite side thereof so as to form said second receiving means, and wherein said second receiving means includes a second notch sized and shaped to receive the second portion of the pupillary margin such that the iris passes under said second flange and said third limb and over said fourth limb and said third flange, and
wherein said ring is formed from a shape memory material, such that said ring is moveable between an undeformed state and a deformed state.

20. The device of claim 19, wherein said ring is manually moveable from its said undeformed state to its said deformed state, and wherein said ring automatically returns to its said undeformed state from its said deformed state.

21. The device of claim 19, wherein said first notch and said second notch are foldable when said ring is in its said deformed state, and wherein said at least three flanges are foldable in a zigzag manner when said ring is in its said deformed state, such that said central space has a smaller area compared to when said ring is in its said undeformed state.

22. The device of claim 21, further comprising a sleeve, wherein said sleeve is sized and shaped to receive said ring in its said deformed state.

23. The device of claim 19, wherein said shape memory material is selected from the group consisting of a self-enlarging material, a self-healing material, a self-reconfiguring programmable material, a biodegradable material, and/or drug-eluting material.

24. The device of claim 19, wherein said ring is closed.

25. The device of claim 19, wherein said ring is opened.

26. The device of claim 16 or 19, further comprising a delivery device, said delivery device including an outer tube sized and shaped to receive said ring in its said deformed state, and a plunger.

27. A device for enlarging a pupil of an eye during surgery, the eye including an iris and the pupil including a pupillary margin adjacent the iris, the device comprising:
a ring at least partially enclosing a central space and having at least three flanges including a first flange, a second flange and a third flange, a first corner portion connectively disposed between said first flange and said second flange and forming first receiving means for receiving a first portion of the pupillary margin, and a second corner portion connectively disposed between said second flange and said third flange and forming second receiving means for receiving a second portion of the pupillary margin, said first corner portion and said second corner portion extending into said central space,
said first corner portion including a first blind end, a first limb connecting said first flange to said first blind end on one side thereof, and a second limb connecting said second flange to said first blind end on an opposite side thereof so as to form said first receiving means, and wherein said first receiving means includes a first notch sized and shaped to receive the first portion of the pupillary margin such that the iris passes over said first flange and said first limb and under said second limb and said second flange, and
said second corner portion including a second blind end, a third limb connecting said second flange to said second blind end on one side thereof, and a fourth limb connecting said third flange to said second blind end on an opposite side thereof so as to form said second receiving means, and wherein said second receiving means includes a second notch sized and shaped to receive the second portion of the pupillary margin such that the iris passes under said second flange and said third limb and over said fourth limb and said third flange.

28. The device of claim 27, wherein said at least three flanges define a first plane.

29. The device of claim 28, wherein said first blind end and said second blind end define a second plane.

30. The device of claim 29, wherein the distance between said first plane and said second plane is within a range of from about 0.1 mm to about 0.5 mm.

31. The device of claim 27, wherein said first flange and said third flange define a first plane, and said second flange defines a second plane.

32. The device of claim 31, wherein the distance between said first plane and said second plane is within a range of from about 0.1 mm to about 0.5 mm.

33. The device of claim 31, wherein said first blind end and said second blind end lie in said first plane, in said second plane, or between said first plane and said second plane.

34. The device of claim 15, 16, 19, 24 or 27 wherein said ring has a substantially hexagonal shape.

35. The device of claim 15, 16, 19, 24 or 27, wherein said ring has a substantially rectangular shape.
